(19) United States
(12) Patent Application Publication
(10) Pub. No.: US 2024/0407718 A1
(43) Pub. Date: Dec. 12, 2024

(54) Systems, Devices and Methods for Dermal Treatments

(71) Applicant: ACOM Labs, Inc., San Francisco, CA (US)

(72) Inventors: Jack Phillip Abraham, Miami Beach, FL (US); Callie Mackenzie Roberts, Martinez, CA (US); Liang Liu, Leander, TX (US); Dehui Kong, Newark, CA (US); Paul F. Bente, IV, Westford, MA (US); Healey Thomas Cypher, San Francisco, CA (US)

(73) Assignee: ACOM Labs, Inc., San Francisco, CA (US)

(21) Appl. No.: 18/694,910

(22) PCT Filed: Sep. 26, 2022
(86) PCT No.: PCT/US2022/077038

§ 371 Date: Mar. 22, 2024

Related U.S. Application Data
(60) Provisional application No. 63/248,396, filed on Sep. 24, 2021.

(51) Int. Cl. A61B 5/00; A61B 90/00; A61M 37/00

(52) U.S. Cl. CPC A61B 5/4839; A61B 5/0077; A61B 5/441; A61M 37/0015; A61B 2090/062; A61M 2037/0023

(57) ABSTRACT

Systems, devices, and methods for dermal treatments are provided. Various systems, devices, and methods provide treatment options with a handheld device, intradermal or subdermal fluid delivery via a needle or microneedle. For fluid delivery, system can include an injector, fluid-filled container, and a needle or hollowed microneedle. A fluid-filled container can be compatibly coupled with a treatment device such to perform the various dermal treatments. Further, fluid delivery systems can be utilized in a number of applications, including medications and supplements for the skin.

### CROSS REFERENCE TO RELATED APPLICATIONS

This application claims priority to U.S. Provisional Application Ser. No. 63/248,396, entitled “Systems, Devices and Methods for Dermal Treatments,” filed Sep. 24, 2021, the disclosures of which are incorporated herein by reference in its entirety.

### TECHNICAL FIELD

The application is generally directed to systems, devices, and methods for dermal treatments, including systems, devices, and methods that utilize machine vision for dermal injections.

### BACKGROUND

Hollowed microneedles are small applicators to deliver fluids, especially vaccines or medications. Microneedles are typically used in transdermal, intraocular, or intracochlear fluidic delivery. Because of their small size, microneedles typically do not cause injury to the site of injection and are generally considered less hazardous than other injection methods, such as a conventional hypodermic needle.

### BRIEF DESCRIPTION OF THE DRAWINGS

The description and claims will be more fully understood with reference to the following figures, which are presented as exemplary embodiments of the disclosure and should not be construed as a complete recitation of the scope of the disclosure.

FIGS. 1A to 1C provide illustrations of handheld treatment devices in accordance with various embodiments of the disclosure.

FIGS. 1D to 1I provide illustrations of handheld treatment devices incorporating machine vision systems in accordance with various embodiments of the disclosure.

FIGS. 2A to 4B provide illustrations of fluid-filled cartridges in accordance with various embodiments of the disclosure.

FIGS. 5A to 5D provide illustrations of component injector systems in accordance with various embodiments of the disclosure.

FIGS. 5E to 5J provide illustrations of component injector systems incorporating a variety of different machine vision systems in accordance with various embodiments of the disclosure.

FIGS. 6A and 6B provide illustrations of cartridges and microneedles as discrete components in accordance with various embodiments of the disclosure.

FIGS. 7 to 9 provide illustrations of mechanics of injector systems with unassisted penetration in accordance with various embodiments of the disclosure.

FIGS. 10 to 12 provide illustrations of mechanics of injector systems with assisted penetration in accordance with various embodiments of the disclosure.

FIG. 13 provides illustrations of mechanics of electromechanical injector systems with assisted penetration in accordance with various embodiments of the disclosure.

FIGS. 14 to 16B provide illustrations of an exemplary injector system in accordance with various embodiments.

FIGS. 17A to 20 provide illustrations of mechanics of an exemplary ejector system in accordance with various embodiments.

FIGS. 21 to 24 provide illustrations of optional features of an exemplary ejector system in accordance with various embodiments.

FIGS. 24 and 25 provide illustrations of an exemplary electromechanical injector system in accordance with various embodiments.

FIGS. 26A to 26C provide illustrations of camera systems utilized within handheld treatment devices in accordance with various embodiments of the disclosure.

FIG. 27A illustrates a papular acne lesion.

FIGS. 27B and 27C illustrate another papular acne lesion an infrared image of the lesion obtained using reflectance confocal microscopy.

FIG. 28 conceptually illustrates a desired injection trajectory for a cystic or papular acne lesion in accordance with an embodiment of the disclosure.

FIGS. 29A and 29B conceptually illustrate image processing processes performed with respect to images acquired by a machine vision system of a handheld treatment device in accordance with various embodiments of the disclosure.

FIG. 30 is a flow chart illustrating a process for detecting, tracking, and administering medication via injection in accordance with various embodiments of the disclosure.

FIG. 31 is a flow chart illustrating a process for detecting, tracking, and administering medication via injection using a Single Shot Detection (SSD) machine learning model to identify and classify acne lesions in accordance with various embodiments of the disclosure.

FIG. 32 is a flow chart illustrating a process for acquiring an image in accordance with embodiments of the disclosure.

FIG. 33 is a flow chart illustrating a process for determining an injection site for administering medication to an acne lesion in accordance with embodiments of the disclosure.

FIG. 34 is a flow chart illustrating a process for performing injection in accordance with embodiments of the disclosure.

FIG. 35 conceptually illustrates an injector processing system in accordance with embodiments of the disclosure.

### DETAILED DESCRIPTION

Turning now to the drawings, systems, devices and methods for dermal care are described, in accordance with various embodiments of the disclosure. Several embodiments are directed towards precise intradermal or subdermal fluidic delivery utilizing a needle or microneedle. In several embodiments, a treatment device is handheld. In various embodiments, the handheld device utilizes a syringe or cartridge filled with fluid for intradermal or subdermal. In various embodiments, the handheld device utilizes a needle or a hollowed microneedle to perform fluidic injection.

In certain embodiments, an intradermal or a subdermal fluidic system utilizes a treatment device and a replaceable fluid-filled container (e.g., syringe or cartridge). The fluid-filled container can store a fluid (e.g., medication or supplement). In certain embodiments, a fluid-filled container is compatibly coupled with a treatment device such that the mechanics of the injector is capable of ejecting the fluid out of the fluid-filled container through a needle or microneedle. In certain embodiments, a needle or microneedle is integrated with the fluid-filled container as a single component. In a number of embodiments, the needle or microneedle and fluid-filled container are each an individual component capable of interlocking together (e.g., Luer lock connector).

In certain embodiments, an intradermal or subdermal delivery system is utilized for delivery of a medication and/or supplement, such as triamcinolone (triamcinolone acetonide or Kenalog), hyaluronic acid, or collagen (or a collagen stimulating agent), which can be used in a variety of treatment applications for skin. For instance, in certain embodiments, an intradermal or subdermal delivery system delivers triamcinolone into an acne lesion as an acne treatment. In certain embodiments, an intradermal or subdermal delivery system delivers hyaluronic acid into the skin. And in certain embodiments, an intradermal or subdermal delivery system delivers collagen and/or a collagen stimulating agent into the skin, which can improve skin elasticity and appearance among other benefits.

In a number of embodiments, an injection system incorporates an imaging system that captures image data utilized to assist and/or automatically perform injection. In many embodiments, the imaging system includes an image acquisition system comprising a camera optics. In several embodiments, the camera optics are capable of resolving images of skin. In certain embodiments, the camera utilizes a macro lens, telecentric optics, and/or periscope optics. In various embodiments, the imaging system utilizes one or more imaging modalities including (but not limited to) capturing color images (e.g., conventional Bayer filter or a Bayer filter including two Red pixels per Blue and Green pixel), multispectral images, near-infrared images, extended color images (color+near-infrared), monochrome images (Black and White or Red), and/or polarized light images. In a number of embodiments, the imaging system includes an illumination source such as (but not limited to) a near-infrared illumination source, and/or a polarized light source. In certain embodiments, the imaging system incorporates two or more cameras for performing depth sensing and/or an illumination system for assisting with depth estimation. As can readily be appreciated, the specific imaging system, the type of cameras, the number of cameras, the imaging modalities, and/or the use of illumination sources are typically dependent upon the requirements of a specific application in accordance with various embodiments of the disclosure.

In several embodiments, the imaging system is part of a machine vision system that utilizes image processes to detect and/or track acne lesions within images captured by the imaging system. In certain embodiments, the machine vision system also performs classification of acne lesions and/or modifies the manner in which treatments are applied to the lesions based upon the classification of the lesion. In certain embodiments, all processing is performed within a handheld treatment device. In a number of embodiments, the handheld treatment device captures images and performs initial processing (e.g. image acquisition and image/video encoding) and transmits the processed image data via wired and/or wireless connection to another device for image processing. In several embodiments, the device that performs image processing is a dedicated device that is a companion to the treatment device. In certain embodiments, the device that performs the image processing is a mobile computational device (e.g., phone, tablet) configured by a software application to process image data captured by a handheld injector device. As can readily be appreciated, the specific hardware configuration and/or image processes performed by a machine vision system utilized in combination with a handheld treatment system are dependent upon the requirements of specific applications in accordance with embodiments of the disclosure. Furthermore, any of the imaging systems and/or machine vision systems described herein can be utilized interchangeably in combination with any of the systems described herein, including (but not limited to) fluid injection systems, without departing from the scope of the invention.

The described systems, devices, and methods should not be construed as limiting in any way. Instead, the present disclosure is directed toward all novel and nonobvious features and aspects of the various disclosed embodiments, alone and in various combinations and sub-combinations with one another. The disclosed systems, devices, and methods are not limited to any specific aspect, feature, or combination thereof, nor do the disclosed systems, devices, and methods require that any one or more specific advantages be present or problems be solved.

Various embodiments of intradermal or subdermal treatment systems and examples of treatment devices and cartridges are disclosed herein, and any combination of these options can be made unless specifically excluded. For example, any of the fluidic delivery devices disclosed, can be used with any type of compatible fluid-filled container, even if a specific combination is not explicitly described. Likewise, the different constructions and features of fluidic delivery systems can be mixed and matched, such as by combining any delivery system type/feature, delivery device type/feature, fluid-filled container, machine vision system, injection processes, processing systems, etc., even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

The terms “proximal” and “distal” as used throughout the description relate to a site of injection. Accordingly, a proximal face or proximal portion of a device is the face or the portion that would be more proximal to a site of injection when an injection is performed. Conversely, a distal face or distal portion of a device is the face or the portion that would be more distal to a site of injection when an injection is performed. Likewise, a proximal movement would be movement of a component in a direction towards a site of injection and a distal movement would be movement of a component in an opposite direction. Although these terms are in relationship to a site of injection, it is to be understood that these terms are used for reference and the site of injection does not need to be present when interpreting the components or movements of the devices and systems described herein.

### Systems and Devices for Dermal Treatments

Various embodiments are directed towards systems and devices for intradermal and/or subdermal treatments. In certain embodiments, an intradermal and/or subdermal treatment system includes a treatment device, a fluid-filled container, and/or a needle/microneedle. Generally, and in accordance with various embodiments described herein, an injector is compatible with a fluid-filled container such that the injector is configured to receive and operatively link with the fluid-filled container. In certain embodiments, when an injector and fluid-filled container are operatively linked, the injector provides mechanics to provide the treatment (e.g., eject fluid from the fluid-filled container through the needle/microneedle). In certain embodiments, a needle or microneedle is integrated with the fluid-filled container as a single component. In certain embodiments, a needle or microneedle and fluid-filled container are each an individual component capable of coupling together (e.g., Luer lock connector).

### Treatment Devices

In certain embodiments, a treatment device is configured to provide mechanics for fluidic ejection out of a fluid-filled container. An injector can operate via mechanical or electromechanical means. In certain embodiments, an injector includes one or more buttons or triggers to initiate and/or drive the mechanical and/or electrical components of the device. In certain embodiments, a button or trigger is mechanically or electrically operatively linked with an internal piston that is operatively linked with cartridge to eject the components out the fluid-filled container and through a needle or microneedle. In certain embodiments, an internal driver system cooperatively interacts with a compression spring, which can help control the flow of fluidic ejection out of the fluid-filled container and/or return the internal driver to an initial position. In certain embodiments, an actuator is operatively linked with an internal driver mechanism that is capable of driving the needle to pierce and situate within the skin for injection. In certain embodiments, an internal driver mechanism is a linear actuator and utilizes one or more of: rotatable threaded rod, a worm gear, a rack and pinion, or a solenoid coil. In certain embodiments, a differential screw mechanism is utilized for fine micron (or less) movements.

In certain embodiments, an electromechanical treatment device includes a power source or battery, such as (for example) a lithium ion battery, however any appropriate power source or batter can be utilized. In certain embodiments, a treatment device includes a computation system, memory, and/or software to provide instructions on performing various tasks of the treatment device. Various task to be performed include (but are not limited to) penetration of skin with a needle, ejection of components out of a replaceable injection system, retrieval of the needle out the skin, provide laser/light, calculation of dosage, calculation of volume to administer, calculation of needle depth for administration, camera image data (live or captured), storage of data, and connection with internet systems or other systems (e.g., Bluetooth, cloud systems, Wi-Fi enabled, cellular data enabled). Data that can be stored within a memory of the treatment device include (but are not limited to) procedure logs, cartridge logs (e.g., type, volume), location logs, dosage logs, and needle depth logs.

In certain embodiments, the needle remains unexposed to the user during the injection process. In certain embodiments, an injection device includes one or more sensors, which can be utilized to sense needle penetration, requisite needle depth, fluid ejection, local pressure, or any other appropriate sensation to be detected. In certain embodiments, an injection device in conjunction with a needle includes a sensor for measuring electrical impedance, which may be used to detect skin contact, needle penetration, and/or needle depth. In certain embodiments, a spacer on the needle system is provided to ensure proper needle penetration and depth.

In certain embodiments, a treatment device includes housing for receiving a fluid-filled replaceable injection system (e.g., cartridge system or syringe system). In certain embodiments, a housing includes a reversible coupling and/or locking mechanism to facilitate the reception of the replaceable injection system. In certain embodiments, a replaceable injection system includes compatible components for coupling and/or locking with the injector. Any appropriate reversible coupling and/or locking mechanism can be utilized, such as (for example) a hook and with receiving groove, a flange, a threaded screw, a twist lock, a ball and lock pin, or any capable combination of coupling and/or locking mechanisms. In certain embodiments, a coupling and/or locking mechanism is reversible such that the replaceable injection system can be displaced from the replaceable injection system, in which displacement can occur prior to and/or after ejection of fluid.

In many embodiments, a treatment device includes a stabilizing feature (e.g., foot or base), which can be utilized to locate and/or stabilize the injector and needle system at a desired location on the skin. In certain embodiments, a stabilizing feature is extended from and connected to an injector system via a connector, which can be any appropriate connector such as a rod and/or strut. In certain embodiments, a stabilizing feature is the proximal face of an injector system housing. In certain embodiments, a stabilizing feature and a needle are cooperatively positioned such that the ejection tip of the needle is capable of extending beyond the stabilizing feature a requisite distance for intradermal or subdermal delivery. Human skin has a depth of approximately 0.5 mm to 5.0 mm, depending on the location. For instance, facial skin is approximately between 1.5 mm and 2 mm, and further varies on facial location (e.g., average thickness of forehead skin is approximately 1.7 mm and average thickness of cheek skin is approximately 1.85 mm). Accordingly, depending on location and use (e.g., intradermal or subdermal injection), in accordance with various embodiments, a needle tip is positioned between 0.5 mm to 5.0 mm beyond the stabilizing feature at time of injection. For uses on facial skin, in accordance with various embodiments, a needle or microneedle tip is positioned approximately between 0.5 mm to 2.0 mm beyond a stabilizing feature at time of injection. In various embodiments, a microneedle or needle tip is positioned approximately 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.5 mm, 3.0 mm, 3.5 mm, 4.0 mm, 4.5 mm, or 5.0 mm beyond the stabilizing feature at time of injection.

In many embodiments, a stabilizing feature includes an element for cooling and/or heating, which may provide a means for mitigating pain or offering comfort to the user during injection. Any appropriate element for providing cooling and/or heating, such as (for example) a coil, a resistor, air vent, vacuum, and/or fan can be utilized. In several embodiments, a vibrator is incorporated into the stabilizing feature or needle, which can also provide a means for mitigating pain or offering comfort to the user during injection.

In certain embodiments, a housing of the injector system partially or entirely conceals the replaceable injection system. In certain embodiments, a housing further conceals a needle. In certain embodiments, a housing can include an orifice (e.g., pinhole) for the needle to be exposed during skin penetration. In certain embodiments, the proximal face of the housing surrounding the orifice can provide stabilizing and/or positioning effect at a desired location on the skin. In certain embodiments, the orifice and a needle are cooperatively positioned such that the ejection tip of the needle is capable of extending beyond the orifice a requisite distance for intradermal or subdermal delivery.

In many embodiments, a treatment device includes one or more imaging modalities (e.g., camera), which may be used to help visualize the treatment and/or record treatment sites images or data. Any appropriate camera can be utilized, including (but not limited to) visible light, polarized light, multispectral, and/or infrared cameras. In some embodiments, the imaging modality is an ultrasound system, which help visualize the injection site and internal tissue structure. In certain embodiments, the imaging modality is positioned proximal to a cartridge such that it is capable of visualizing the treatment site and/or procedure.

When mounted to an injection device, a camera can be proximate the treatment site. In order to accommodate comparatively small focal distances, a variety of optical systems can be utilized in various embodiments of the disclosure. In several embodiments, a lens barrel incorporating a macro lens is utilized. In several embodiments, folded optics and/or periscope optics are utilized. In a number of embodiments, a telecentric lens systems is utilized to provide a large depth of field. As can readily be appreciated, any optical system appropriate to the requirements of a specific treatment can be utilized in accordance with embodiments of the disclosure. In certain embodiments, a light is utilized to enhance camera and/or user visualization. In many embodiments, a laser is utilized to help guide a user to the proper injection site. In a number of embodiments, a laser works in conjunction with a camera to provide precise treatment. In several embodiments, an illumination system is utilized that enhances features (e.g. polarized light) and/or enables subcutaneous imaging (e.g. an infrared light source). As can readily be appreciated, any illumination system appropriate to the requirements of specific applications can be utilized in combination with imaging systems in accordance with embodiments of the disclosure.

In several embodiments, a treatment device includes a means for providing feedback to ensure proper treatment. In certain embodiments, an injector system includes a means for providing feedback for when the replaceable injection system is securely within the device. In certain embodiments an injector system includes a means for providing feedback for when the replaceable injection system is not securely within the device. In certain embodiments, an injector system includes a means for providing feedback for when the injector system is ready for use. In certain embodiments, an injector system includes a means for providing feedback for when the injector system is actively providing treatment. In certain embodiments, an injector system includes a means for providing feedback for when the injector system has finished providing treatment. Any appropriate means for providing feedback can be utilized, including (but not limited to) a white light, a colored light, covering or uncovering of mechanical features on the device with and without use of color, tactile feedback such as vibration, and an audible sound.

### Fluid-filled Containers

Several embodiments are directed towards interchangeable fluid-filled containers to be utilized in conjunction with a treatment device. Any compatible cartridge, syringe, or other fluid-filled container can be used with the device. In various embodiments, a fluid-filled container is compatible with the treatment device. In certain embodiments, a fluid-filled container includes a reversible coupling and/or locking mechanism to facilitate the reception of the cartridge into a receiver of the treatment device. In certain embodiments, a fluid-filled container includes compatible components for coupling and/or locking with the treatment device. Any appropriate reversible coupling and/or locking mechanism can be utilized, such as (for example) a hook and with receiving groove, a flange, a threaded screw, a twist lock, a ball and lock pin, or any capable combination of coupling and/or locking mechanisms. In certain embodiments, a coupling and/or locking mechanism is reversible such that the cartridge can be displaced from the treatment device, in which displacement can occur prior to and/or after use of the cartridge components.

Many embodiments are directed towards fluid-filled containers to be utilized in conjunction with a treatment device. In certain embodiments, a fluid-filled container is a sealed container with fluid therein, which can be hermetically sealed. Any appropriate volume of fluid can be utilized. In various embodiments, a fluid-filled container contains approximately 0.01 cc to 10 cc. In various embodiments, a fluid-filled container contains approximately 0.01 cc, 0.05 cc, 0.1 cc, 0.15 cc, 0.2 cc, 0.25 cc, 0.3 cc, 0.35 cc, 0.4 cc, 0.45 cc, 0.5 cc, 0.55 cc, 0.6 cc, 0.65 cc, 0.7 cc, 0.75 cc, 0.8 cc, 0.85 cc, 0.9 cc, 0.95 cc, 1.0 cc, 1.5 cc, 2.0 cc, 2.5 cc, 3.0 cc, 3.5 cc, 4.0 cc, 4.5 cc, 5.0 cc, 5.5 cc, 6.0 cc, 6.5 cc, 7.0 cc, 7.5 cc, 8.0 cc, 8.5 cc, 9.0 cc, 9.5 cc, or 10.0 cc.

In certain embodiments, a fluid-filled container is for limited-use, such as single-use fluid-filled container or multi-use fluid-filled container. In various embodiments, a fluid-filled container contains fluid for multiple injections. In certain embodiments, a fluid-filled container is disposable after fluid ejection. In certain embodiments, a fluid-filled container contains a plunger or is under pressure to facilitate ejection of fluid out of the container and through a needle, microneedle or other tip.

In certain embodiments, a plunger of a fluid-filled container is capable of operatively linking with an internal driver of a treatment device (e.g., injector device). In certain embodiments, an internal driver of a treatment device is capable of contacting a face of fluid-filled container (e.g., a face opposite of a needle) such that the driver can operatively push a plunger of the fluid-filled container, resulting in ejection of liquid out of the container. In certain embodiments, a fluid-filled container is capable of operatively linking with an internal drive mechanism of a treatment device such that the internal drive mechanism can move an injection system in an axial direction away and/or toward from a center portion of the treatment device. In certain embodiments, movement of a fluid-filled container via an internal drive mechanism of a treatment device simultaneously moves the ejection tip of a needle or microneedle and/or toward from a center portion of the injector, such that the internal drive mechanism operatively drives the needle or microneedle to pierce and insert into skin. In certain embodiments, an internal drive mechanism of an injector moves the ejection tip of a needle or microneedle to the requisite position beyond a stabilizing feature.

Several embodiments are directed towards fluid-filled containers, especially fluids for use in dermatological treatment and/or supplement. Fluids to be used within a fluid-filled container include (but are not limited to) medicine, supplements, triamcinolone, hyaluronic acid, collagen, or other liquids.

### Needles, Microneedles and Ejection Tips

Several embodiments are directed to the use of needles, microneedles and ejection tips to expel component out of a component-containing cartridge. A needle or microneedle can be used for injecting a component while an ejection tip can provide topical treatment of a component. In certain embodiments, a needle, microneedle or ejection tip is operatively linked with fluid-filled container such that fluid within the cartridge can be expelled from the container via the needle, microneedle or tip. In certain embodiments, a needle, microneedle or ejection tip extends from a face of the container (e.g., a face opposite of a face that interacts win an internal piston of an injector). In certain embodiments, a needle, microneedle or ejection tip is integrated with the cartridge such that the microneedle/tip and cartridge are a single component. In certain embodiments, a microneedle/tip and cartridge are each an individual component capable of fitting together to ensure flow out of the cartridge and through the microneedle or tip. Any appropriate means for fitting a microneedle or tip with a cartridge can be utilized, such as (for example) a Luer lock system or a gasket.

In various embodiments, one or more microneedles is operatively linked with a fluid-filled container such that fluid can be ejected out of the container via the one or more microneedles. In certain embodiments, a single microneedle is operatively linked with a fluid-filled container. In certain embodiments, a plurality of microneedles is operatively linked with a fluid-filled container, which can be arranged in an array, a regular pattern (e.g. circle), an irregular pattern, or any other configuration.

In some embodiments, a needle or microneedle has ability to provide a cooling effect, a heating effect, or a microvibration effect. Accordingly, a means to provide cooling, heating, or microvibration is operatively linked with the needle to provide the function. Any appropriate means for providing needles with cooling, heating, or microvibration capability can be utilized.

In certain embodiments, one or more needles or microneedles are veiled or concealed, which may be desirable to prevent harm to a user from the needle or microneedle or for preventing damage to the needle or microneedle. Any appropriate means of veiling or concealing one or more needle or microneedles can be utilized. In certain embodiments, a covering is situated surrounding a needle or microneedle. In certain embodiments, a covering is rigid and/or firm material. In embodiments utilizing a rigid and/or firm covering, the covering can unveil or reveal the microneedle through an orifice or pinhole, which can happen as it is advanced or prior to advancement into the injection site. In certain embodiments, a covering is collapsible and/or puncturable material such that a needle or microneedle is unveiled or revealed by the covering collapsing and/or the needle or microneedle puncturing through the covering. Puncturable material include (but are not limited to) rubber, neoprene, PTFE, ePTFE and metallic foil. In certain embodiments, after ejection of fluid out of the cartridge via a needle or microneedle, the needle or microneedle is re-veiled or re-concealed. In certain embodiments, a rigid or firm covering ejects outwards from the housing and covers the needle or microneedle after injection.

### Machine Vision Systems

A handheld treatment device can incorporate and/or be in communication with a machine vision system including an imaging system and a processing system such as (but not limited to) a machine vision processing system. As discussed above, any of a variety of imaging systems can be utilized as appropriate to the requirements of specific applications. In many embodiments, the imaging system is utilized to capture image data within a field of view of the imaging system. In several embodiments, the field of view of the imaging system images a region in which the device can administer a treatment (e.g., intra- or subdermal injection of fluid).

In several embodiments, the machine vision system controls the acquisition of image data and analyzes image data to detect region of interest. In several embodiments, regions of interest are regions that contain a detected dermal condition. In a number of embodiments, a region of interest can contain any dermal condition of interest in a particular application. A dermal condition can be a skin ailment, a lesion (e.g., acne lesion), dermal injury, keloid, wrinkle, dermal abnormality, discoloration, or any other dermal condition that is detectable and capable of being treated by a treatment system as described herein.

In many embodiments, detection is performed using a set of one or more rules that analyze pixels of acquired image data to determine whether one or more dermal conditions (e.g., acne lesions) are present. An additional set of rules can be utilized to classify a detected dermal condition and/or a machine learning model including (but not limited to) a support vector machine, a cascade of classifiers, and/or a neural network (e.g., a convolutional neural network). In several embodiments, a classifier is utilized that is trained using a supervised learning process (e.g., a process in which a set of labeled images are utilized to train the classier) to detect whether a region of interest contains a dermal condition. In certain embodiments, a process is utilized that both evaluates in real time whether regions of interest contain a dermal condition, detect specific features of the dermal condition (e.g., pilosebaceous unit localization of an acne lesion), and/or perform classification of any detected dermal condition.

In a number of embodiments, detection is performed using a neural network system trained to generate a set of features and including layers that perform detection within different regions of interest. In this way, the neural network can efficiently generate a single set of features that are utilized to perform detection in parallel across a number of size and aspect ratio regions of interest. In various embodiments, separate networks can be trained to perform detection of different classes of dermal condition. In certain embodiments, separate networks can be trained to perform detection of different classes of acne lesions (e.g., cystic acne, papulopustular acne, open comedomes and/or closed comedomes). In this way, the networks can be evaluated in parallel to enable real time detection and classification of dermal conditions. In a number of embodiments, a neural network such as the Single Shot MultiBox Detector described in Liu, Wei, et al. “Ssd: Single shot multibox detector.” European conference on computer vision. Springer, Cham, 2016 (the disclosure of which including the disclosure related to the training and use of an SSD machine learning model in image processing applications is hereby incorporated by reference in its entirety) is utilized. While specific machine learning models are described above, it should be readily appreciated that any of a variety of machine learning models that can be utilized for image processing applications can be utilized as appropriate to the requirements of specific applications, including (but not limited to) convolutional neural networks (CNNs) such as Alexnet, ResNet, VGGNet, and/or Inception, in accordance with embodiments of the disclosure.

In certain embodiments, real time processing of acquired image data is achieved by performing an initial detection of a dermal condition and then tracking the condition in subsequent images in a sequence of acquired images. In this way, a less computationally intensive tracking process can be utilized to track dermal conditions. In several embodiments, processes including (but not limited to) optical flow and/or structure from motion techniques are utilized to track features of a detected dermal condition. In a number of embodiments, a feature detection process is performed to detect features that can then be tracked. Features that can be detected include (but are not limited to) Scale Invariant Feature Transform (SIFT) features, log polar SIFT features, SIFT-Histogram of Gradient (SIFT-HOG) features, and/or skin lesion specific bundles of features such as (but not limited to) the features described in Upadhyay, Pawan Kumar, and Satish Chandra. “An improved bag of dense features for skin lesion recognition.” Journal of King Saud University-Computer and Information Sciences (2019) (the disclosure of which including the disclosure related to the detection of skin specific features is hereby incorporated by reference in its entirety). As can readily be appreciated, any of a variety of processes appropriate to the requirements of specific applications can be utilized to perform feature tracking. In many embodiments, the ability to track the features of a condition enable detection of when the handheld treatment device is appropriately positioned to deliver treatment to a treatment site.

In a number of embodiments, the treatment site is determined based upon the classification of the dermal condition and/or the specific treatment to be administered. In certain embodiments, the handheld treatment device provides audio, tactile, and/or visual feedback to assist the user to position the handheld treatment device in an appropriate orientation relative to the treatment site to deliver the treatment. In a number of embodiments, the handheld treatment device automatically initiates the treatment when oriented correctly. In several embodiments, the handheld treatment device provides feedback to the user to manually initiate treatment when the handheld treatment device is oriented correctly. In certain embodiments, the treatment involves injection and the handheld treatment device includes sensors that monitor the depth of penetration of the injection and/or the volume of fluid administered during the injection.

### Exemplary Systems and Devices

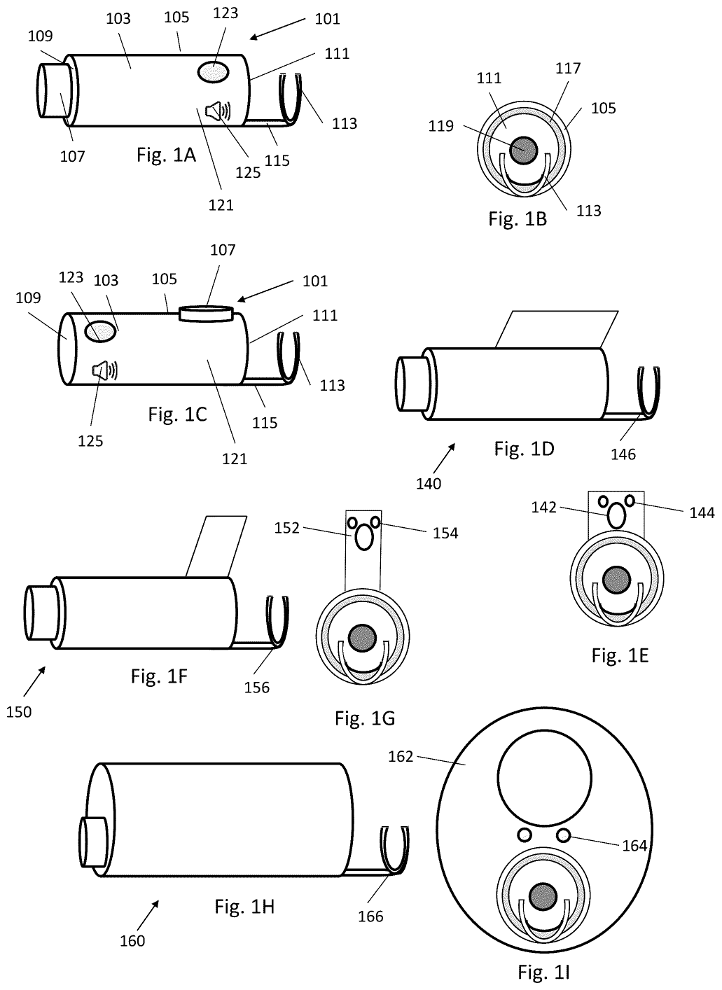

Turning now to FIGS. 1A to 1C, examples of a treatment device are provided, in accordance with various embodiments of handheld treatment devices. As can be seen in FIG. 1A, a treatment device 101 with injector capabilities can include a body 103 with an external covering 105 that covers an internal piston. Device 101 can include a button 107 that can initiate and/or drive the mechanics of the internal piston and an internal driver. As shown, button 107 is on a face 109 opposite of a face 111 that couples with a component-filled cartridge (not shown). Device 101 can further include a stabilizing and/or position foot 113 that extends away from body 103 via a strut connector 115. As shown, stabilizing and/or positioning foot 113 extends away from face 111 capable of coupling with a fluid-filled cartridge. Device 101 can also optionally include a light feedback indicator 123 and a sound feedback indicator 125 to provide feedback of one or more of the following: securement of the cartridge, ready for use, active engagement of treatment, finished providing treatment, or any other appropriate feedback.

Provided in FIG. 1B is a view of device 101 showing face 111 that couples with a component-filled cartridge. Face 111 includes a coupling portion 117 for coupling the injector with a component-filled cartridge. Face 111 further shows an internal piston 119 that moves in axial direction away and towards from a central portion 121 of the body.

FIG. 1C provides another example device 101 in which button 107 extends from a curved face of cylindrical body 103.

In a number of embodiments, the handheld treatment device incorporates an imaging system and/or an illumination system. In several embodiments, the imaging system includes one or more cameras or other imaging modality (e.g., ultrasound). In a number of embodiments, the illumination system includes one or more illumination sources.

With specific reference to FIGS. 1D to 1I, various embodiments of handheld treatment devices that include a camera system and multiple illumination sources are illustrated. As is discussed further below, camera systems utilized within handheld treatment devices can incorporate any of a number of different optical systems that are capable of capturing in focus images of skin during use of the handheld treatment device. With specific regard to FIGS. 1D and 1E, a handheld treatment device 140 including a camera system having telecentric optics 142 is shown. When in use, the camera system has a field of view of skin adjacent the positioning foot 146. With specific regard to FIGS. 1F and 1G, a handheld treatment device 150 including a camera system having periscope 152 is shown. When in use, the camera system has a field of view of skin adjacent the positioning foot 156. With specific regard to FIGS. 1H and 1I, a handheld treatment device 160 including a camera system having macro optics 162 is shown. When in use, the camera system has a field of view of skin adjacent the positioning foot 166. As can readily be appreciated any of a variety of camera systems can be utilized as appropriate to the requirements of specific applications to resolve images of regions of skin containing dermal ailments (e.g., acne lesions) in accordance with embodiments of the disclosure.

The camera systems and the illumination sources of the handheld treatment devices illustrated in FIGS. 1D to 1G are shown as contained within a housing extending from the side of the handheld treatment device, which can be attached thereon or integrated within. However, any of a variety of housing form factors can be utilized as appropriate to the requirements of particular applications. A handheld treatment device including a cylindrical housing in accordance with an embodiment of the disclosure is illustrated in FIGS. 1H and 1I. While the discussion of FIGS. 1D to 1I above focuses on the imaging and illumination systems that can be incorporated within handheld treatment devices, the handheld treatment devices shown in FIGS. 1D to 1I also include components similar to those found in the handheld treatment devices discussed above with references to FIGS. 1A to 1C. Furthermore, the handheld treatment devices shown in FIGS. 1D to 1I should be understood as being capable of implementing any of the components and/or features of any of the handheld treatment devices described herein.

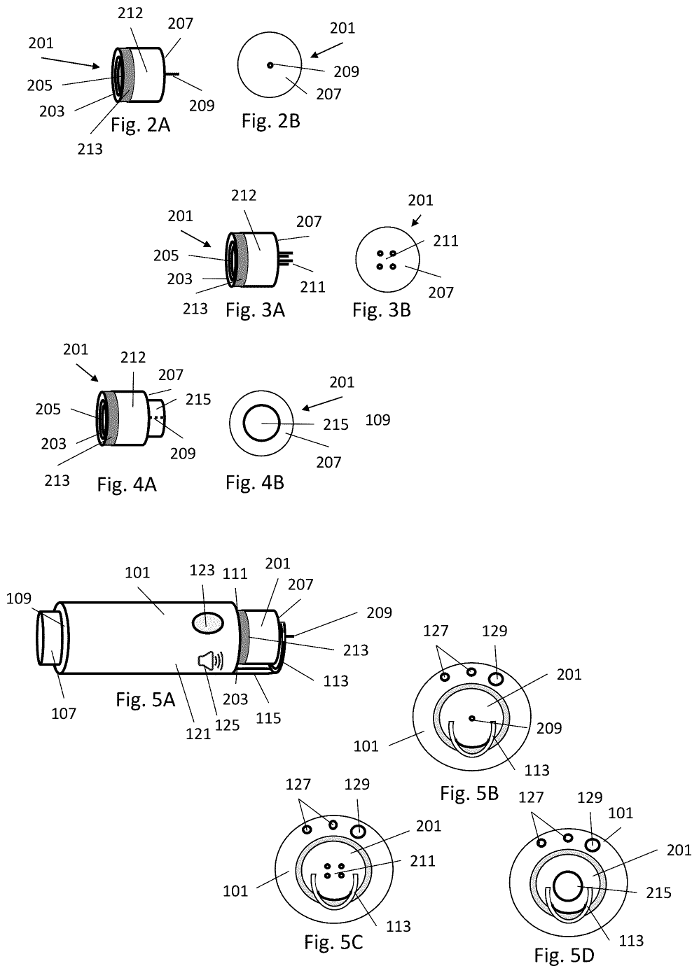

FIGS. 2A to 4B provide various examples of component-filled cartridges 201, in accordance with various embodiments. As can be seen within these figures, a cartridge can include a face 203 capable of coupling with an injector, including a central portion 205 that can interact with an internal piston of the injector. Opposite of face 203 capable of coupling with an injector is a face 207 with one or more microneedles. As seen in FIGS. 2A and 2B, a single microneedle 209 can be utilized. Alternatively, as seen in FIGS. 3A and 3B, a plurality of microneedles 211 can be utilized, which can be in for the form of an array (e.g., 2×2), a pattern (e.g., a circle), or an irregular pattern, each microneedle having a microneedle ejection tip 210. Within cartridge 201 is a plunger 213 and a fluid-filled portion 212 that stores fluid until it is ejected from the cartridge. Plunger 213 can interact with central portion 205 of face 203, which can interact with an internal piston of the injector such that the plunger can moved in axial direction away from face 203 and towards the one or more microneedles 209/211.

FIGS. 4A and 4B provide an example of a covering 215 that veils and/or conceals one or more needles (only a single needle 209 is portrayed as dashed lined), in accordance with various embodiments. Covering 215 can surround the one more needles to provide concealment. The covering can include one or more pinholes (not shown) that can allow for exposure of the one or more concealed needles as they advance through the pinholes. Alternatively, the cover can be of a puncturable material such that the one or more needles can be exposed by puncturing through material as they are advanced.

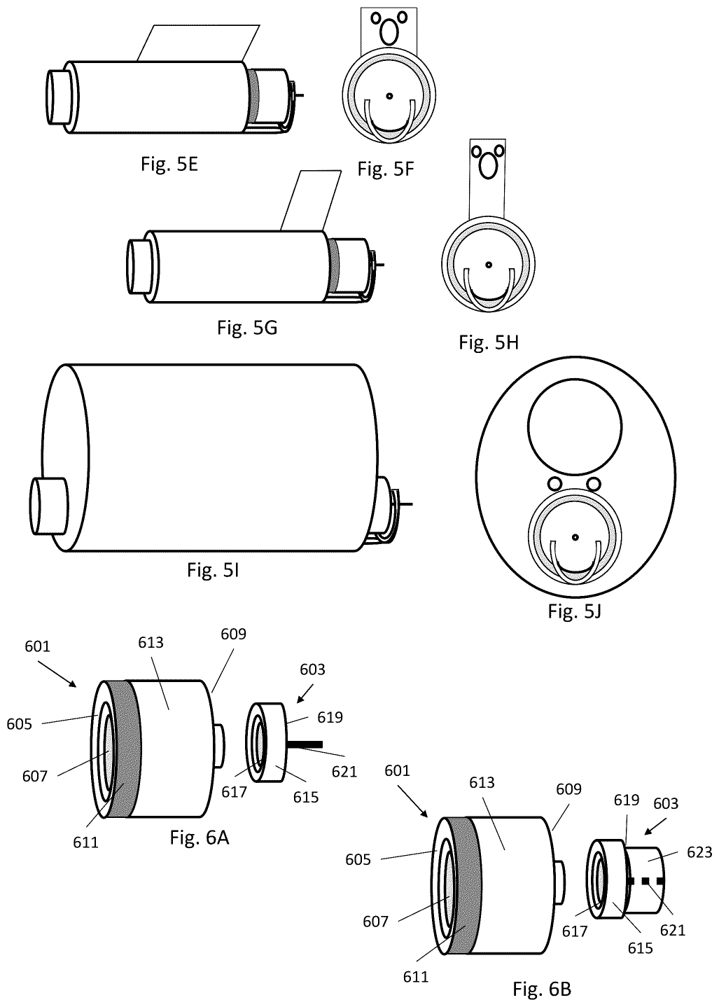

Provided in FIGS. 5A to 5G are the exemplary treatment device 101 of FIG. 1A operatively linked with the exemplary component-filled cartridges 201 of FIGS. 2A to 4B, in accordance with various embodiments. FIGS. 5E to 5J illustrate handheld treatment devices similar to those shown in FIGS. 1D to 1I, but configured to be operatively linked with the exemplary component-filled cartridges 201 of FIGS. 2A to 4B, in accordance with additional embodiments. As can be seen in FIG. 5A, cartridge 201 can situate within device 101 such that face 203 of cartridge 201 is in contact with face 111 of the device 101. Central portion 205 of face 203 of the cartridge interacts with internal piston 119 of the device 101. Further, microneedle 209 extends in a direction away from device 101 and is positioned such that microneedle ejection tip 210 is advanced beyond foot 113. As discussed previously, the precise position the microneedle tip in reference to the foot depends on the desired type of delivery (e.g., intradermal or subdermal) and the thickness of the skin at the injection site. FIGS. 5B to 5D show a view of face 207 of cartridge 201 situated in device 101. Device 101 can also optionally include a light feedback indicator 123 and a sound feedback indicator 125 to provide feedback of one or more of the following: securement of the cartridge, ready for use, active engagement of treatment, finished providing treatment, or any other appropriate feedback. Further, device 101 can incorporate one or more cameras 127 and visualization light 129, which may be used to assist and/or record use of the device and cartridge.

As can readily be appreciated cartridges similar to those discussed above with reference to FIGS. 5A to 5D can also be utilized in a similar manner within the embodiments illustrated within FIGS. 5E to 5J that incorporate imaging and/or illumination systems.

FIGS. 6A and 6B provide examples of a cartridge unit 601 and microneedle unit 603 as individual units that can be assembled together, in accordance with various embodiments. Cartridge 601 includes a face 605 capable of coupling with an injector, including a central portion 607 that can interact with an internal piston of the injector. Opposite of face 605 capable of coupling with an injector is a face 609 capable of coupling with a microneedle unit 603. Within cartridge 601 is a plunger 611 and a component-filled portion 613 that stores fluid until it is ejected from the cartridge. Plunger 611 can interact with central portion 607 of face 605, which can interact with an internal piston of the injector such that the plunger can moved in axial direction away from face 605 and towards the microneedle assembly 603.

Microneedle unit 603 includes a base 615 with a face 617 capable of coupling with face 609 of cartridge 601. The coupling can be any appropriate coupling that allows for adequate fluid from the cartridge and into the microneedle unit, such as (for example) a Luer lock or gasket. Opposite of face 617 is a face 619 with a microneedle 621 that extends away from the microneedle unit base 615. Although not shown, a microneedle unit can include a plurality of microneedles, which can be formed into an array or any other pattern. As shown in FIG. 6B, a microneedle unit 603 can include a covering 623 that veils and/or conceals one or more needles (only a single needle 621 is portrayed as dashed lined). Covering 623 can surround the one or more needles to provide concealment. The covering can include one or more pinholes (not shown) that can allow for exposure of the one or more concealed needles as they advance through the pinholes. Alternatively, the cover can be of a puncturable material such that the one or more needles can be exposed by puncturing through material as they are advanced.

### Injector Systems

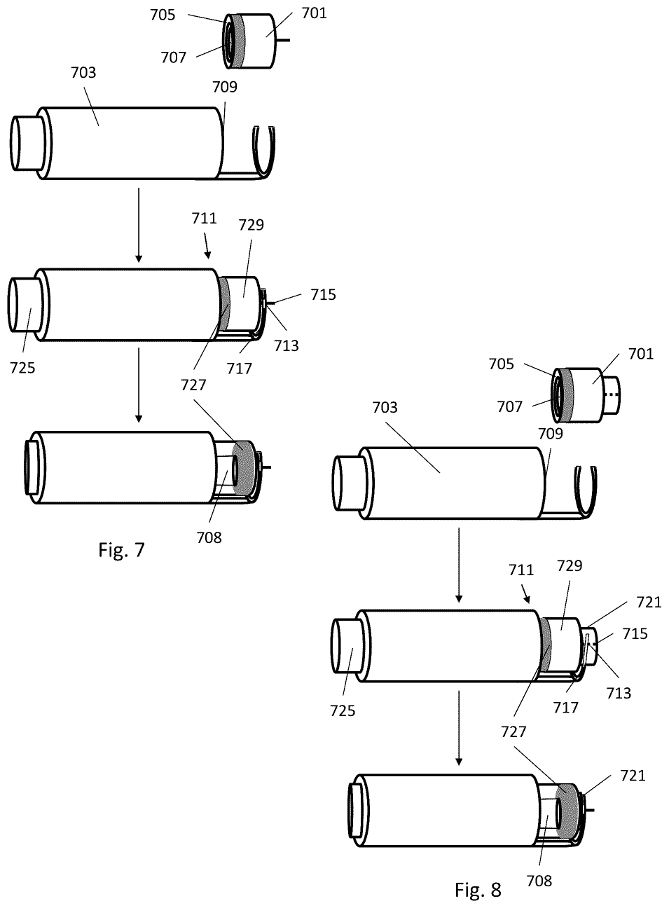

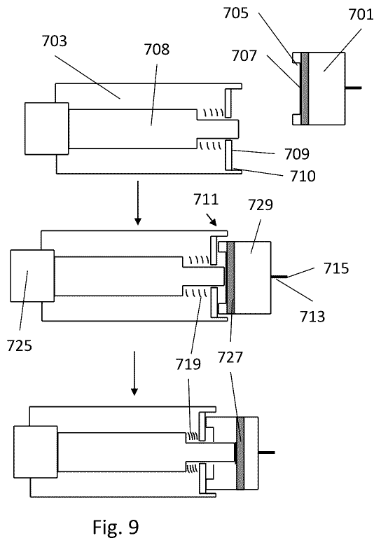

Provided in FIGS. 7 to 9 are examples of microneedle injector systems with unassisted skin penetration, in accordance with various embodiments. A cartridge 701 is loaded onto a treatment device 703. Cartridge 701 includes a face 705 with a central portion 707 cooperatively couples with a face 709 and internal piston 708 of treatment device 703. The outer portion 710 of face 709 includes a reversible coupling and/or locking mechanism to facilitate the reception of face 705 of cartridge 701. Coupling of cartridge 701 with treatment device 703 results in an injector system 711. Assembled injector system 711 includes a microneedle 713 extends in a direction away from the treatment device 703. Assembly of injector system 711 results in a microneedle ejection tip 715 that is appropriately positioned in relationship to a foot 717 such that the ejection tip extends beyond the foot a requisite distance for intradermal or subdermal injection. Note, for sake of simplicity and explanation, FIG. 9 does not show a foot but it can be assumed one is present on the assembled system. Microneedle 713 can be concealed utilizing a covering 721 as shown in FIG. 8. Although not shown, other cartridges (e.g., superficial ablation tip, light emitting diode), can be coupled into the injector in a similar manner.

Assembled microneedle injector system 711 can be used for intradermal or subdermal injection of liquid or solvent. A user can penetrate skin with microneedle ejection tip 715 at a desired location, moving microneedle 713 perpendicular to the surface of the skin and penetrating into the skin until foot 717 rests upon the outer surface of the skin, resulting in the microneedle tip having the requisite depth for proper intradermal or subdermal injection. With proper depth, injector system 711 can inject liquid into the skin. Covering 721 can be pierceable or include a pinhole such that microneedle 713 can be exposed to penetrate the user's skin. As shown in FIG. 8, covering 721 is collapsible such that as the needle penetrates the user's skin, it collapses until the needle reaches the requisite depth for proper intradermal or subdermal injection, resulting in a collapse covering 723. As noted herein, a covering can be retractable and/or removable instead of being collapsible.

Injector system 711 utilizes a spring 719 that is operative with internal piston 708 to facilitate liquid ejection. A button 725 is utilized to move piston 708 in an axial direction towards cartridge 701. As piston 708 moves in the axial direction, the piston interacts with center portion 707 of face 705, pushing the center portion in the axial direction and towards microneedle 713. Center portion 707 interactions with a plunger 727 to displace liquid within a liquid containing portion 729 of cartridge 701, resulting in liquid passing through microneedle 713 and out of ejection tip 715. After injection of liquid into the skin, microneedle 713 can be removed the skin. Multi-use cartridges can be utilized for multiple injections and the steps to inject liquid into another desired location can be repeated. After cartridge 701 is spent, it can be removed and disposed and treatment device 703 can be reused with a subsequent cartridge.

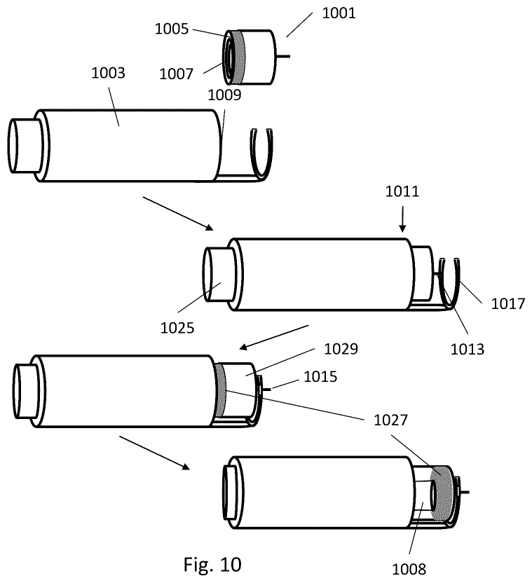

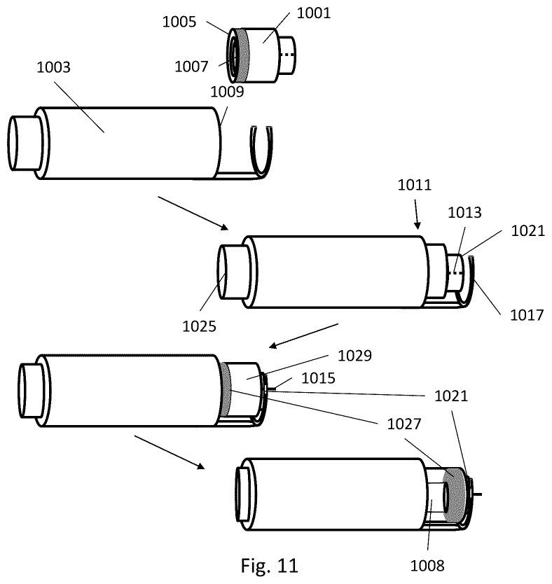

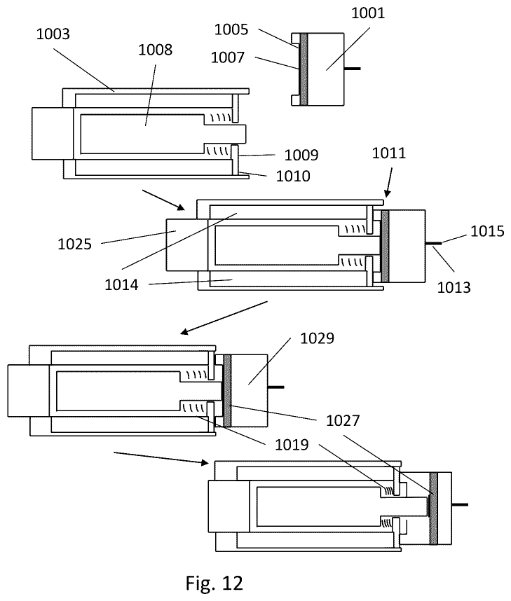

Provided in FIGS. 10 to 12 are examples of microneedle injector systems with assisted skin penetration, in accordance with various embodiments. A cartridge 1001 is loaded onto a treatment device 1003. Cartridge 1001 includes a face 1005 with a central portion 1007 cooperatively couples with a face 1009 and internal piston 1008 of treatment device 1003. The outer portion 1010 of face 1009 includes a reversible coupling and/or locking mechanism to facilitate the reception of face 1005 of cartridge 1001. Outer portion 1010 further includes an operative link with an internal driver 1014 that facilitates assisted skin penetration. Coupling of cartridge 1001 with treatment device 1003 results in an injector system 1011. Assembled injector system 1011 includes a microneedle 1013 extends in a direction away from the treatment device 1003. Assembly of injector system 1011 results in a microneedle ejection tip 1015 that is slightly recessed from a requisite distance for intradermal or subdermal injection. As shown in FIGS. 10 and 11, ejection tip 1015 in slightly recessed in relationship to a foot 1017, which can allow for a user to position injector system 1011 utilizing foot 1017 prior to penetrating skin with microneedle 1013. Note, for sake of simplicity and explanation, FIG. 12 does not show a foot but it can be assumed one is present on the assembled system. Microneedle 1013 can be concealed utilizing a covering 1021 as shown in FIG. 11. Although not shown, other cartridges (e.g., superficial ablation tip, light emitting diode), can be coupled into the injector in a similar manner.

Assembled microneedle injector system 1011 can be used for intradermal or subdermal injection of liquid. Once a user positions injector system 1011, the system can assist the user to penetrate their skin with microneedle ejection tip 1015 at a desired location. The user can push a button 1025 to initiate internal driver 1014, thus moving microneedle 1013 perpendicular to the surface of the skin and penetrating into the skin until ejection tip 1015 requisite depth for proper intradermal or subdermal injection. As shown in FIG. 12, internal driver 1014 is a one or more rigid outer members, such as one or more struts or sheath encircling inner piston 1008. Button 1025 can push internal driver 1014 in an axial direction towards cartridge 1001, resulting in the outer portion 1010 of face 1009 pushing the cartridge in the axial direction. As cartridge 1001 moves axially, microneedle 1013 penetrates the user skin until ejection tip 1015 reaches proper depth. With proper depth, injector system 1011 can inject liquid into the skin. Covering 1021 can be pierceable or include a pinhole such that microneedle 1013 can be exposed to penetrate the user's skin. As shown in FIG. 11, covering 1021 is collapsible such that as the needle penetrates the user's skin, it collapses until the needle reaches the requisite depth for proper intradermal or subdermal injection, resulting in a collapse covering 1023. As noted herein, a covering can be retractable and/or removable instead of being collapsible.

Injector system 1011 utilizes a spring 1019 that is operative with internal piston 1008 to facilitate liquid ejection. Button 1025 is utilized to move piston 1008 in an axial direction towards cartridge 1001. Alternatively, a second button can be utilized to facilitate movement of the piston in the axial direction. As piston 1008 moves in the axial direction, the piston interacts with center portion 1007 of face 1005, pushing the center portion in the axial direction and towards microneedle 1013. Center portion 1007 interactions with a plunger 1027 to displace liquid within a liquid containing portion 1029 of cartridge 1001, resulting in liquid passing through microneedle 1013 and out of ejection tip 1015. After injection of liquid into the skin, microneedle 1013 can be removed the skin. Multi-use cartridges can be utilized for multiple injections and the steps to inject liquid into another desired location can be repeated. After cartridge 1001 is spent, it can be removed and disposed and treatment device 1003 can be reused with a subsequent cartridge.

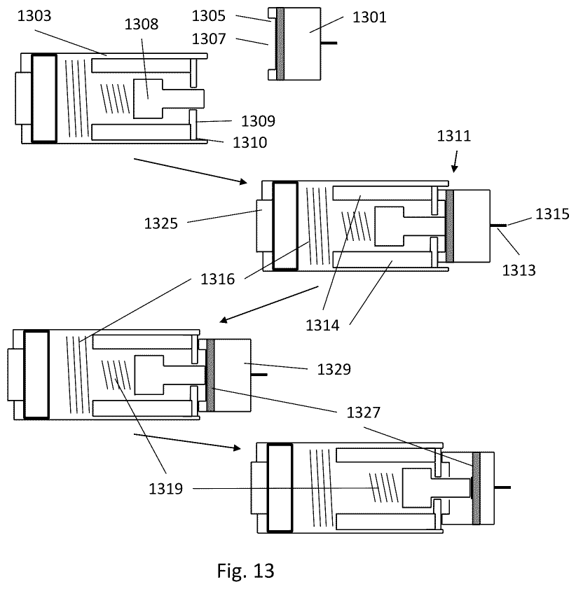

Provided in FIG. 13A is an example of an electromechanical microneedle injector system with assisted skin penetration, in accordance with various embodiments. A cartridge 1301 is loaded onto an electromechanical treatment device 1303. Cartridge 1301 includes a face 1305 with a central portion 1307 cooperatively couples with a face 1309 and internal piston 1308 of treatment device 1303. The outer portion 1310 of face 1309 includes a reversible coupling and/or locking mechanism to facilitate the reception of face 1305 of cartridge 1301. Outer portion 1310 further includes an operative link with an internal driver 1314 that facilitates assisted skin penetration. Coupling of cartridge 1301 with treatment device 1303 results in an injector system 1311. Assembled injector system 1311 includes a microneedle 1313 extends in a direction away from the treatment device 1303. Assembly of injector system 1311 results in a microneedle ejection tip 1315 that is slightly recessed from a requisite distance for intradermal or subdermal injection. Note, for sake of simplicity and explanation, FIG. 13A does not show a foot but it can be assumed one is present on the assembled system. Microneedle 1313 can be concealed utilizing a covering. Although not shown, other cartridges (e.g., superficial ablation tip, light emitting diode), can be coupled into the injector in a similar manner.

Assembled microneedle injector system 1311 can be used for intradermal or subdermal injection of liquid. Once a user positions injector system 1311, the system can assist the user to penetrate their skin with microneedle ejection tip 1315 at a desired location. The user can push a button 1325 to initiate rotatable threaded rod 1316 that is operatively linked with internal driver 1314, which can be powered by a battery 1320 or other power source. Initiation of internal driver 1314 moves microneedle 1313 perpendicular to the surface of the skin and penetrating into the skin until ejection tip 1315 requisite depth for proper intradermal or subdermal injection. Internal driver 1314 is a one or more rigid outer members, such as one or more struts or sheath encircling inner piston 1308. Rotatable threaded rod 1316 can push internal driver 1314 in an axial direction towards cartridge 1301, resulting in the outer portion 1310 of face 1309 pushing the cartridge in the axial direction. As cartridge 1301 moves axially, microneedle 1313 penetrates the user skin until ejection tip 1315 reaches proper depth. With proper depth, injector system 1311 can inject liquid into the skin.

Injector system 1311 utilizes a second rotatable rod 1319 that is operative with internal piston 1308 to facilitate liquid ejection. Button 1025 is utilized to initiate rotation of rod 1319 to move piston 1308 in an axial direction towards cartridge 1301. As piston 1308 moves in the axial direction, the piston interacts with center portion 1307 of face 1305, pushing the center portion in the axial direction and towards microneedle 1313. Center portion 1307 interactions with a plunger 1327 to displace liquid within a liquid containing portion 1329 of cartridge 1301, resulting in liquid passing through microneedle 1313 and out of ejection tip 1315. After injection of liquid into the skin, microneedle 1313 can be removed the skin. Multi-use cartridges can be utilized for multiple injections and the steps to inject liquid into another desired location can be repeated. After cartridge 1301 is spent, it can be removed and disposed and treatment device 1303 can be reused with a subsequent cartridge.

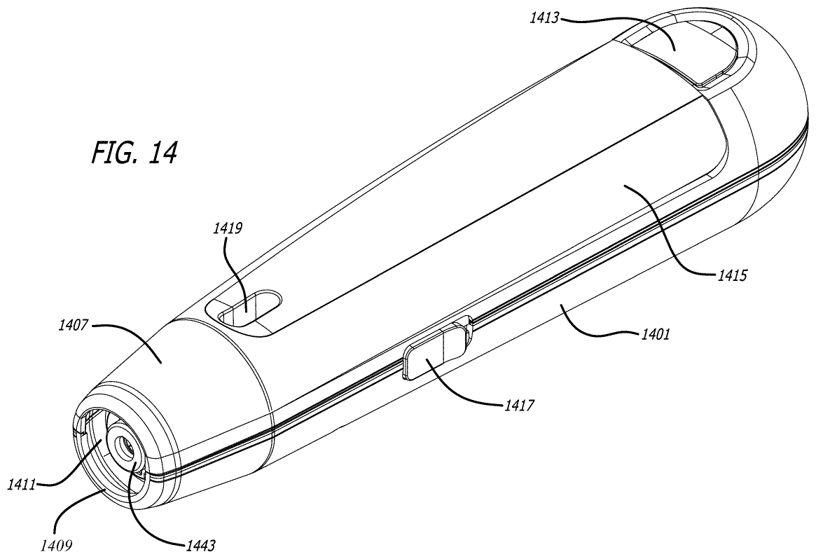

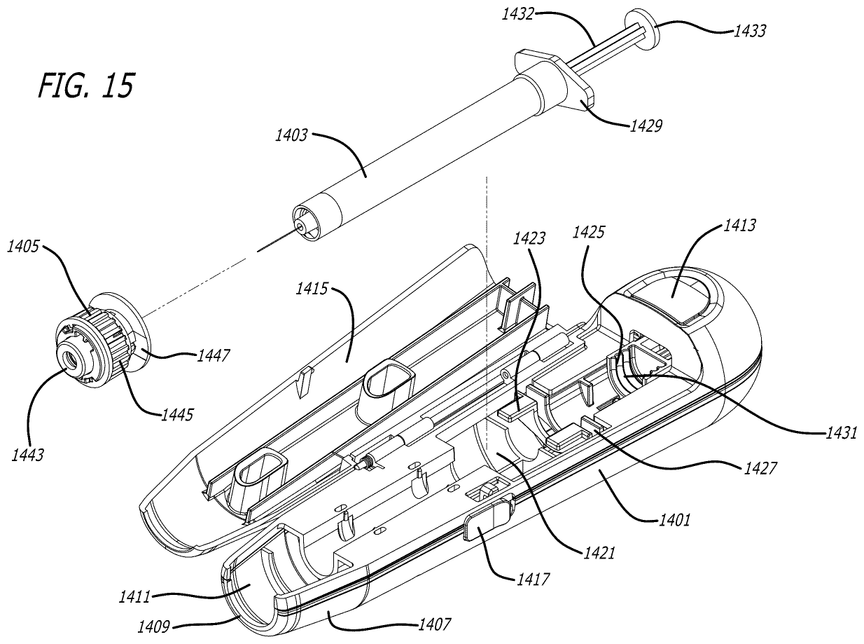

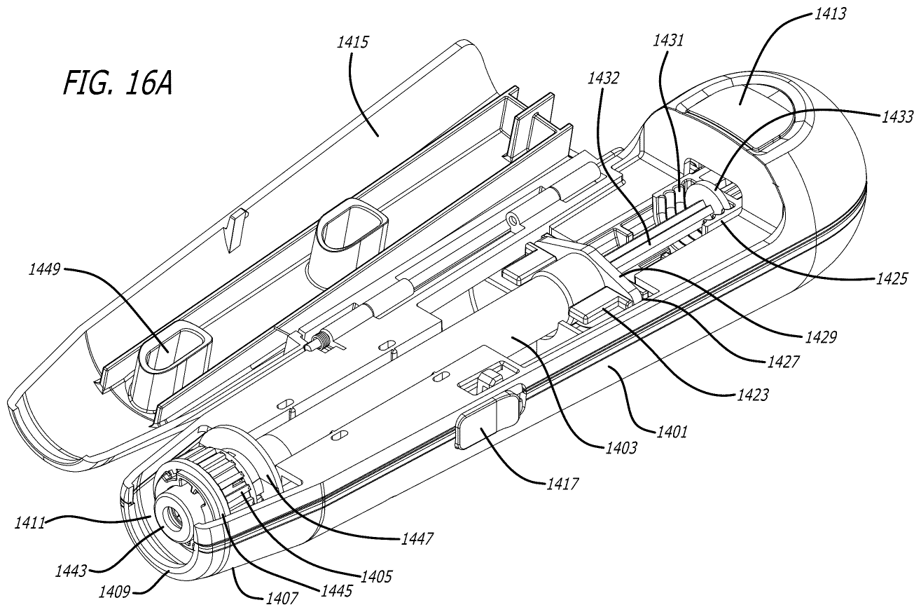

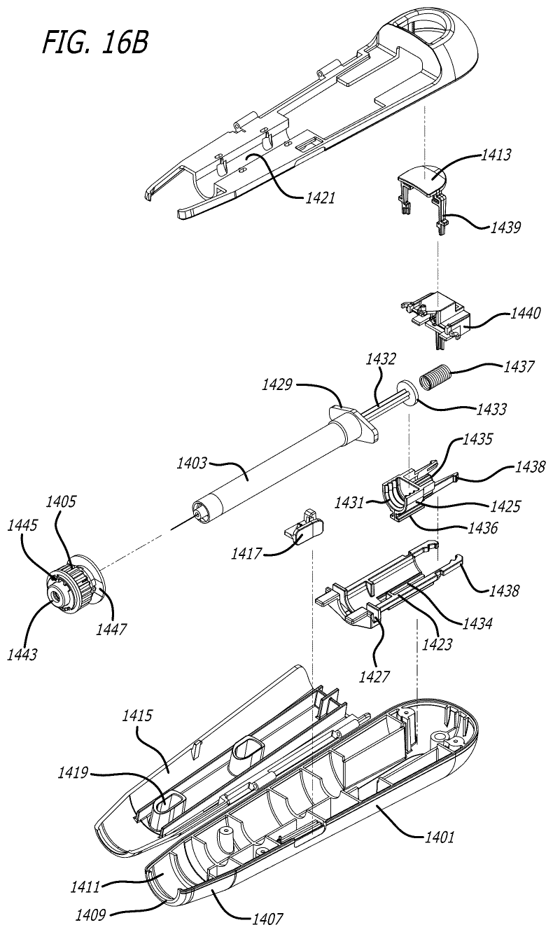

Provided in FIGS. 14 to 16B is an exemplary injector system for performing intradermal or subdermal injection of a liquid. The system as shown comprises a housing compartment 1401, a fluid-filled syringe 1403, and a needle assembly 1405. FIG. 14 shows the housing compartment 1401 in its closed state. FIG. 15 shows housing 1401 with lid 1415 in the open position. And FIGS. 16A and 16B show fluid-filled syringe 1403 and needle assembly 1405 installed within housing compartment 1401. It is to be understood that the exemplary system depicted in FIGS. 14 to 16B can utilize any of the camera systems described herein. Specifically, the exemplary system can include a camera system having telecentric optics (see FIGS. 1D, 1E, 5E and 5F), a camera system having a periscope (see FIGS. 1F, 1G, 5G, and 5H), or a camera system having macro optics (see FIGS. 1H, 1I, 5I, and 5J). Generally, these camera systems can be implemented by attaching the camera system housing and components on the side of the housing intradermal or subdermal injection or integrated within the housing.

Housing compartment 1401 contains a proximal portion 1407 that is associated with needle assembly 1405 and provides a proximal face 1409 for contacting with skin when performing injection and an orifice 1411 to allow for injection. Housing compartment 1401 further contains a button 1413 for actuating an injection mechanism. A lid 1415 is provided with a latch 1417 that can open to allow for situating fluid-filled syringe 1403 and needle assembly within housing 1401. A window 1419 is provided for viewing the fluid-filled syringe 1403 and volume of fluid therein.

Needle assembly 1405 can connect with fluid-filled syringe 1403 by any appropriate means, such as a Luer lock. A connected fluid-filled syringe 1403 and needle assembly 1405 can be received by the housing 1401, which can contain a contoured indentation 1421 that conforms to the connected fluid-filled syringe 1403 and needle assembly 1405. Within housing 1401 are a syringe flange holder 1423 and plunger retainer 1425. Flange holder 1423 contains an indentation 1427 that is contoured to the shape of the syringe flange 1429 such that the syringe flange and snugly fit within the indentation. Likewise, plunger retainer 1425 contains a plurality of indentations 1431 each of which are contoured to the shape of plunger grip 1433 at the distal end of plunger 1432 such that the plunger grip and fit within one of the indentations. The plurality indentations allow for flexibility of plunger grip location which may vary depending on the volume of fluid within the syringe and the dose of fluid to be expelled.

Flange holder 1423 and plunger retainer 1425 each are movable in either a proximal direction or a distal direction along a central axis. Flange holder 1423 contains a groove 1434 that cooperates with slider 1436 of plunger retainer 1425. Groove 1434 and slider 1436 allow for plunger retainer 1425 capability to slide in either direction along the groove, independent of movement of flange holder 1423. Syringe flange holder 1423 and plunger retainer 1425 connect with each other via a latch 1438, and the plunger retainer contains a driver 1435 in operable connection with a compressed spring 1437 to provide the driving force for the injection mechanism. Spring 1437 is held in place by a distal base 1440 at the distal end of the system. Button 1413 contains two inward protruding struts 1439 that hold driver 1435 in place and spring 1437 in a compressed state. When button 1413 is pressed inward, inward protruding struts 1439 move along with the button in an inward direction, releasing the compression of spring 1437 to provide a force for driver 1435 to drive flange holder 1423 and plunger retainer 1425 in direction toward proximal portion 1407 along the central axis.

Needle assembly 1405 comprises a needle 1441. In some implementations, the needle assembly further comprises a protective cover 1443, an outer cylinder 1445, and an actuator ring 1447. Protective cover 1443 can prevent exposure of needle 1441 before and after injection, preventing the ability of the needle to prick or cause injury when not performing injection. Outer cylinder 1445 can provide a means to grip needle assembly 1405 and can further help facilitate ensuring protective cover 1443 adequately covers needle 1441. Actuator ring 1447 can unlock a mechanism for re-covering of protective cover 1443 over needle 1441 after injection. It should be understood that the needle assembly can be a standard needle without a protective cover, an outer cylinder, and an actuator ring. In some implementations, when used in housing compartment 1401 the length of the needle is such that when performing fluid injection, the tip of the needle extends beyond proximal face 1409 to control needle depth at the site of injection. In some implementations, the needle has a length such that controlled intradermal injection can be performed.

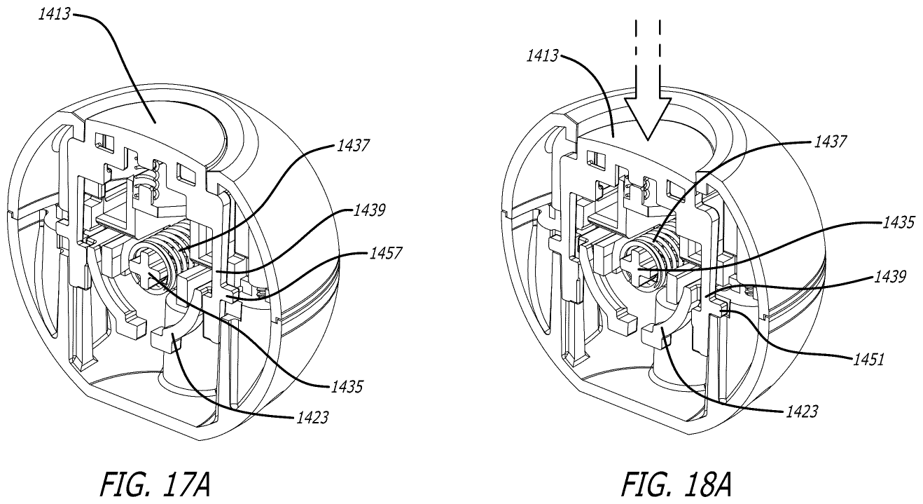

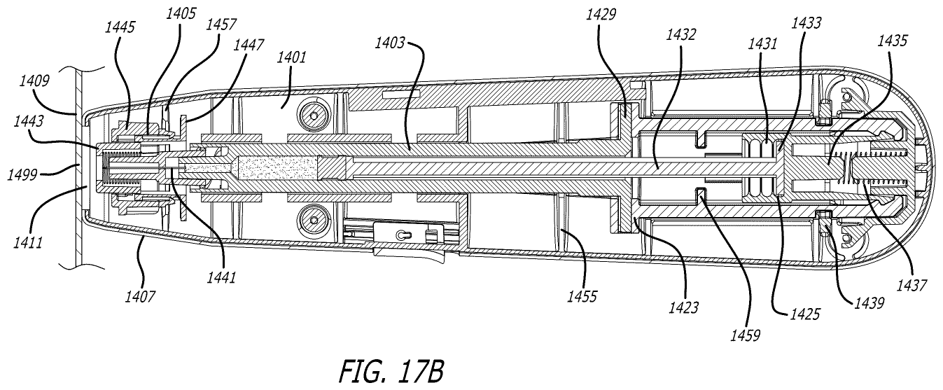

FIGS. 17A to 20 provide an example of various states of a system performing intradermal or subdermal liquid delivery. As described in reference to FIGS. 14 to 16B, the system comprises a housing compartment 1401, a fluid-filled syringe 1403, and a needle assembly 1405. Needle assembly 1405 is attached to fluid-filled syringe 1403 and fitted within housing compartment 1401. Specifically, syringe flange 1429 is situated within flange holder 1423 and plunger grip 1433 is situated within plunger retainer 1425, securing fluid-filled syringe 1403 within the housing.

FIGS. 17A and 17B show the system in an initial state in which the system is loaded with fluid-filled syringe 1403 and needle assembly 1405 and ready to perform the injection mechanism. In this state, fluid-filled syringe 1403, needle assembly 1405, flange holder 1423, and plunger retainer 1425 are in a distal position along the central axis. Fluid-filled syringe 1403 contains a volume of fluid that is greater than the amount the amount to be injected. The relative position of plunger retainer 1425 along a central axis at the initial state determines the injection dose and thus the position can be adjusted at this initial state to control injection dose. Needle 1441 is within cover 1443 and actuator ring 1447 is in an initial closed state. Proximal face 1409 is in contact with a skin surface 1449 at a site to receive injection.

Button 1413 is in an initial outward state such that inward protruding struts 1439 maintain spring 1437 in a compressed state, which is in physical connection with flange holder 1423. Inward protruding struts 1439 each contain a protruding portion 1451 that is in contact with flange holder 1423, maintaining the flange holder and plunger retainer 1425 in place and spring 1437 in the compressed state.

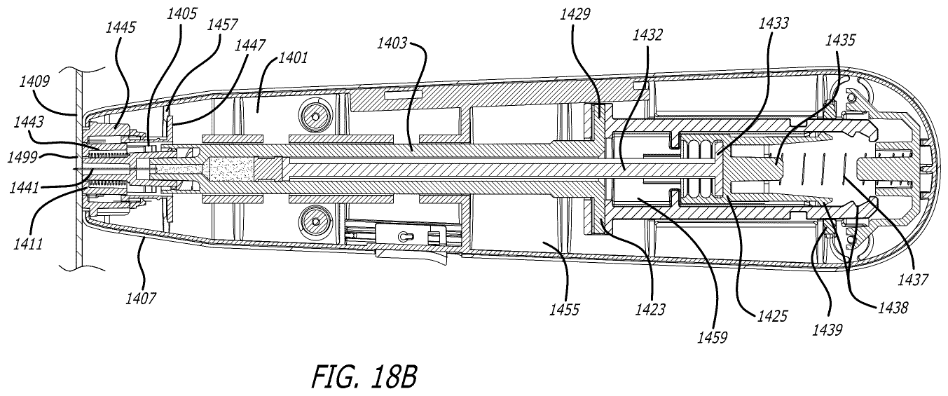

FIGS. 18A and 18B show the initiation of the injection mechanism, resulting in needle 1441 piercing into skin surface 1449. Button 1413 is an actuator of the injection mechanism that when compressed inward 1453 results in the protruding portion 1451 of inward protruding struts 1439 to move further inward such that they are no longer in contact with flange holder 1423. Compressed spring 1437 decompresses moving driver 1435 in a proximal direction along a central axis. Utilizing the spring force, driver 1435 drives flange holder 1423 and plunger retainer 1425 in a proximal direction along a central axis. This results in fluid-filled syringe 1403 and needle assembly 1405 to slide in the proximal direction toward skin surface 1449. As needle assembly 1405 comes into contact with skin surface 1449, cover 1443 contacts the skin and stops its movement, allowing needle 1441 to move proximally past the cover as it pierces into the skin surface. Flange holder 1423 continues to move in the proximal direction until it reaches a flange holder hard stop 1455, halting the proximal movement of the flange holder. The flange holder hard stop also controls the placement of needle assembly 1405 in relationship to skin surface 1449, allowing for precise subdermal or intradermal positioning of the needle tip. Further, as needle assembly 1405 moves in the proximal direction, actuator ring 1447 hits an actuator ring hard stop 1457 opening the actuator ring (i.e., the ring is now held in a more distal position in relation to outer cylinder 1445). By opening the actuator ring, cover 1443 will be allowed to re-cover needle 1441 when the needle is removed from skin surface 1449.

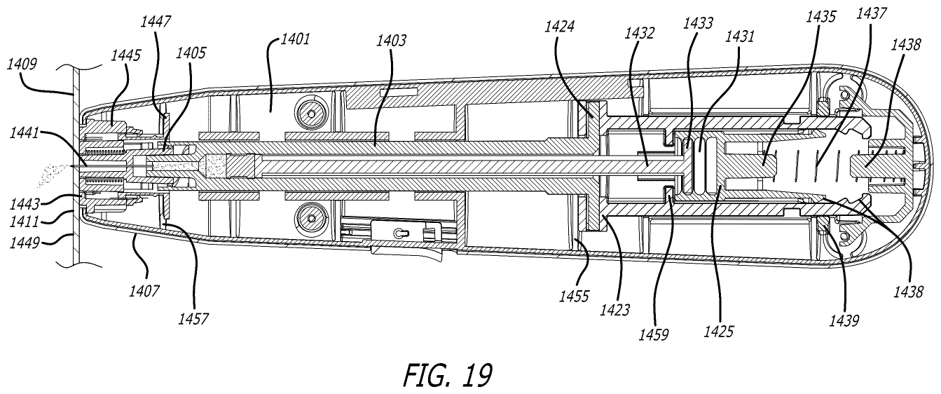

FIG. 19 shows the delivery of a dose of fluid from fluid-filled syringe 1403 through needle 1441 into skin surface 1449. With flange holder 1423 at the flange holder hard stop 1455 position, the flange holder can no longer move in the proximal direction causing latch 1438 to disengage. At this point, driver 1435 continues to drive plunger retainer 1425 in the proximal direction via groove 1434 within flange holder 1423 and slider 1436 of the plunger retainer. As plunger retainer 1425 moves proximally and fluid-filled syringe 1403 remains in place, plunger 1432 is pushed proximally to displace the dose of fluid to be administered, which passes through needle 1441 and into skin surface 1449. Plunger retainer 1425 moves in the proximal direction until it comes into contact with plunger retainer hard stop 1459, which is firmly connected to flange holder 1423. Accordingly, the distance between the position of plunger retainer 1425 and the position of plunger retainer hard stop 1459 controls the fluid dose. When plunger retainer 1425 reaches plunger retainer hard stop 1459, the delivery of fluid into skin 1449 is completed.

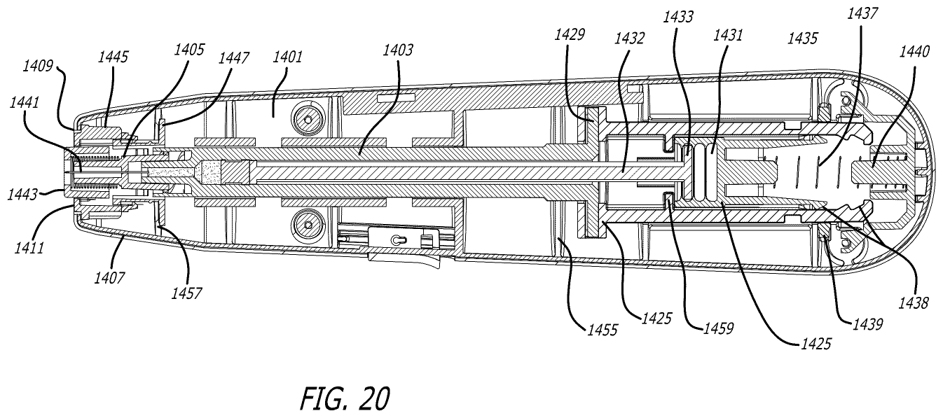

FIG. 20 shows the removal the injector system from skin surface 1449, resulting in cover 1443 covering needle 1441. The fluid-filled syringe 1403, needle assembly 1405, flange holder 1423, and plunger retainer 1425 are in a proximal position. At this time, lid 1415 can be opened up for removal of fluid-filled syringe 1403 and needle assembly 1405. To reset the injector system, plunger retainer 1425 and flange holder 1423 can be slide distally to their initial distal position. Button 1413 can be reset to its outward initial position such that protruding portion 1451 of inward facing struts 1439 hold driver 1435 and compressed spring 1437 in its initial position.

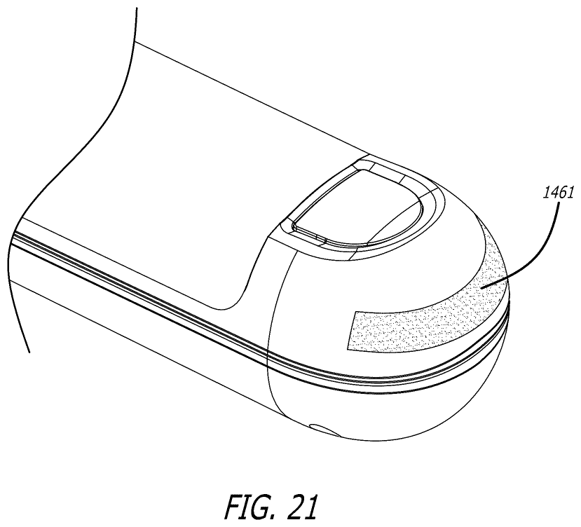

FIG. 21 shows the distal end of an injector system having an optional light indicator 1461, which can be battery powered (not shown). Light indicator 1461 can provide various different status indications to help assist a user. Status indications can provide a user notice of operability, readiness of use, warnings, errors, injection status, and battery power status. Various optional status indications that can be utilized include (but are not limited to) on, unloaded, properly loaded, improperly loaded, ready for injection, injection in progress, injection complete, failure to complete injection, and low battery power. The various status indications can be signaled by various light colors and/or patterns of light (e.g., blinking, flashing, wave-like).

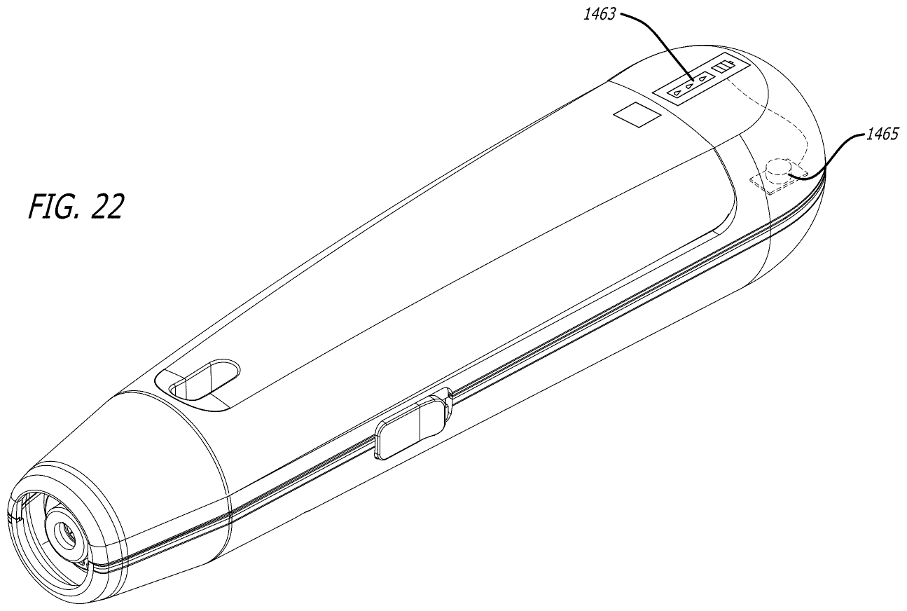

FIG. 22 shows the distal end of an injector system having an optional led screen 1463, which can be powered by a battery 1465. Led screen 1463 can provide various different status indications to help assist a user. Status indications can provide a user notice of operability, readiness of use, warnings, errors, injection status, and battery power status. Various optional status indications that can be utilized include (but are not limited to) on, unloaded, properly loaded, improperly loaded, ready for injection, injection in progress, injection complete, failure to complete injection, and low batter. The various status indications can be signaled by representative icons, color indicators, or script.

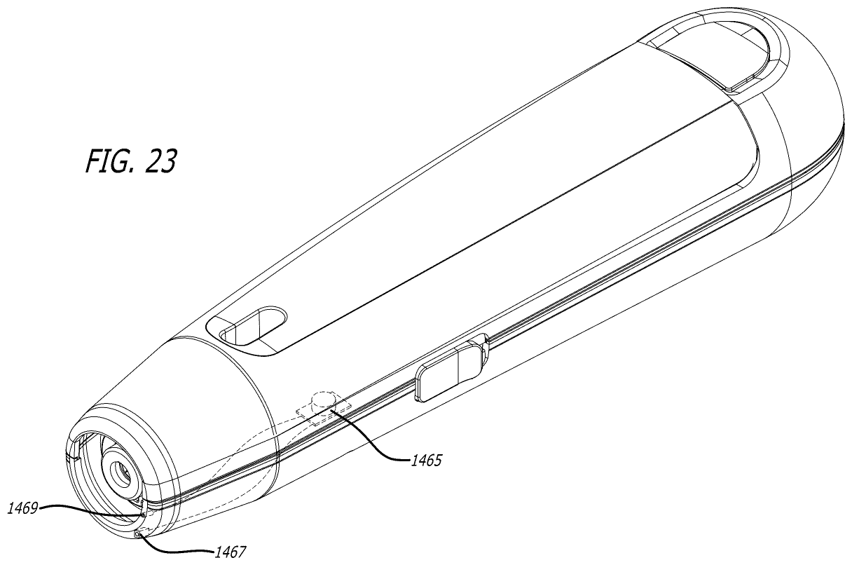

FIG. 23 shows the proximal end of an injector system having an optional camera 1467 and an optional laser light 1469, each of which can be powered by a battery 1465. Camera 1467 can take images of the lesion to be treated. Laser light 1469 can help assist a user to properly locate the injector system onto the lesion to be treated.

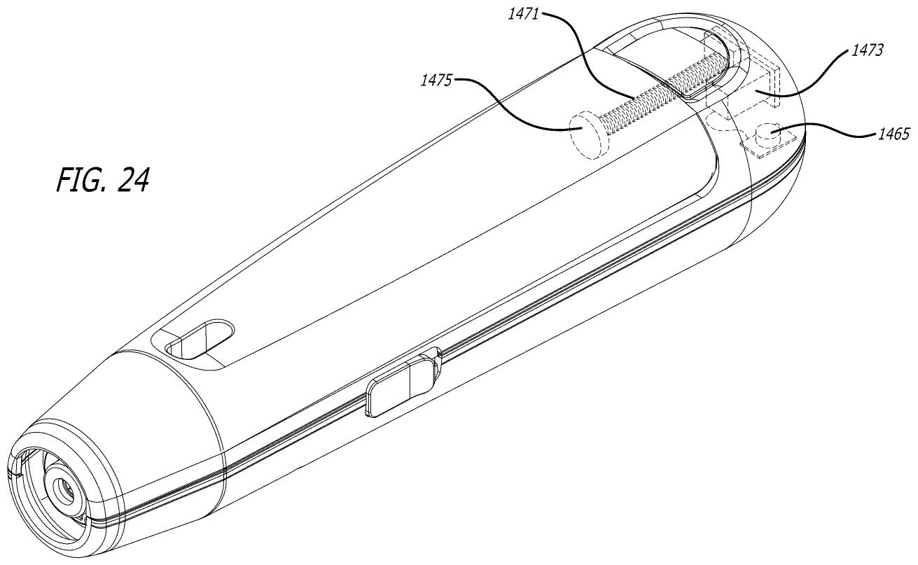

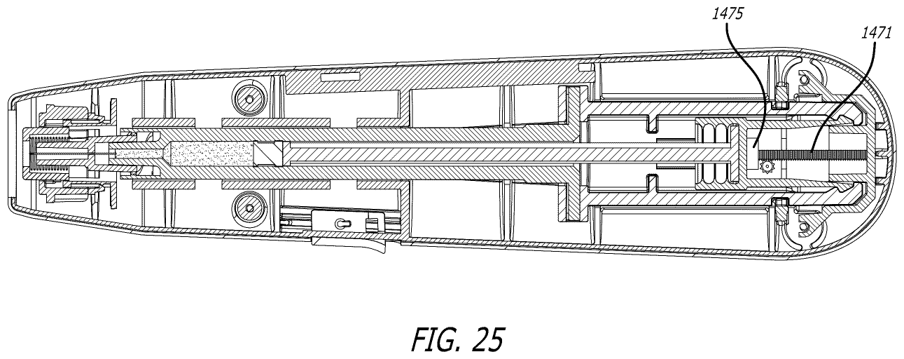

FIGS. 24 and 25 show an electromechanical injector system having an electromechanical linear actuator 1471, a motor 1473 and battery 1465 for powering the motor and linear actuator. Any linear actuator can be utilized, such as (for example) a threaded screw, a worm gear, a rack and pinion, or a solenoid coil. The electromechanical injector shown here can have all the same components and features and have the same mechanistic function of the spring-powered injector system of FIGS. 14 to 20 with the following modifications. Instead of a compressed spring, the electromechanical injector system utilizes a linear actuator 1471 (e.g., as shown a rack and pinion), which can be driven by motor 1473. The linear actuator 1471 includes a head 1475 that is in connection with a driver. Notably, the driver is slightly modified to be compatible with head 1475 instead of a compressed spring. Button 1413 does not hold a spring in a compressed state, but instead initiates the motor to turn the pinion, causing head 1475 and the driver to move in the proximal direction. The movement of the driver in the proximal direction can proceed to drive the injection mechanism as shown in FIGS. 18A to 20 and described in accompanying text. The linear actuator 1471 can also perform the reset (i.e., pull driver in distal direction), instead of manually resetting the injector system.

It is to be understood that the exemplary system depicted in FIGS. 24 and 25 can utilize any of the camera systems described herein. Specifically, the exemplary system can include a camera system having telecentric optics (see FIGS. 1D, 1E, 5E, 5F and 26A), a camera system having a periscope (see FIGS. 1F, 1G, 5G, 5H, and 26B), or a camera system having macro optics (see FIGS. 1H, 1I, 5I, 5J, and 26C). Generally, these camera systems can be implemented by attaching the camera system housing and components on the side of the housing intradermal or subdermal injection or integrated within the housing. Further, a processing system can direct the electromechanical injector system to perform treatment in accordance with the machine vision systems as described herein. Accordingly, a camera system can image a dermal condition and the machine vision system can identify the dermal ailment and direct the electromechanical device to perform the appropriate treatment.

### Imaging Systems

A variety of cameras and/or imaging devices can be utilized in handheld treatment devices in accordance with various embodiments of the invention. A challenge that can be encountered when incorporating a machine vision system within a handheld treatment device is the requirement to resolve images of skin at a potentially short focal distance. As is discussed further below, a variety of optical systems can be utilized to acquire images of skin proximate the end of a handheld treatment device in accordance with various embodiments of the invention.

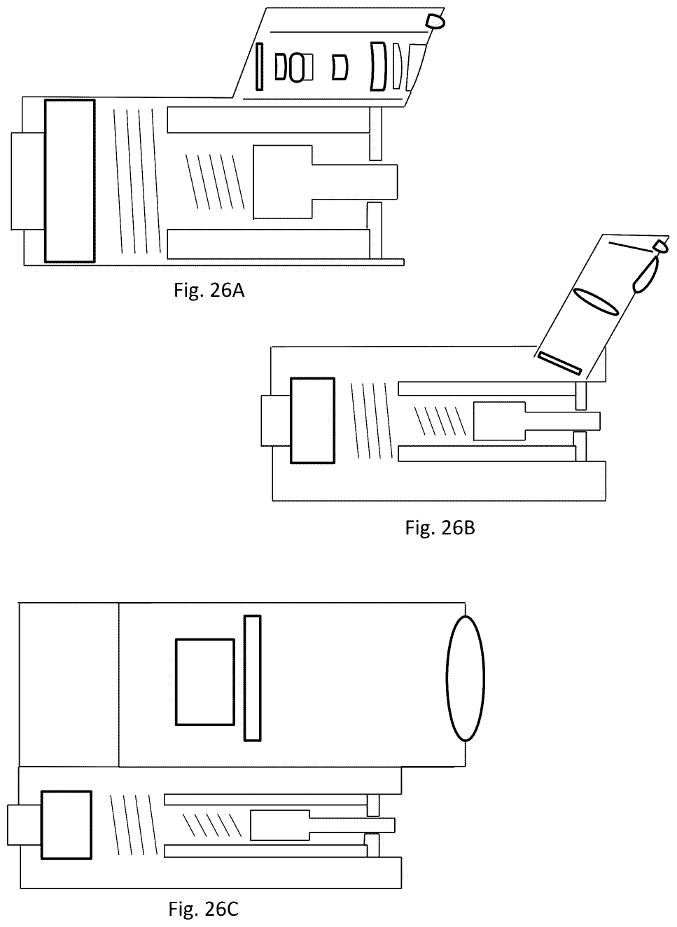

In a number of embodiments, a camera system incorporating telecentric optics is utilized. A telecentric lens is typically considered to be a compound lens that can provide an orthographic view of a subject. Stated another way, use of a telecentric lens leaves the image size unchanged with object displacement, provided the object stays within a certain range often referred to as a depth of field (or telecentric range). Use of a telecentric lens can provide a benefit that the focus and ability of a machine vision system (see discussion below) to detect and/or classify acne lesions is independent of the distance of the handheld treatment device from a user's skin within the depth of field of the telecentric optics. An injector device incorporating an imaging system including a camera with telecentric optics in accordance with an embodiment of the invention is illustrated in FIG. 26A.

In many embodiments, periscope optics are utilized to redirect light within an imaging system to enabled increased separation between the camera aperture and an image sensor. In this way, a greater distance can be established between a camera module and the scene being imaged (e.g. the skin of the user). An injector device incorporating an imaging system including a camera with periscope optics in accordance with an embodiment of the invention is illustrated in FIG. 26B.

In several embodiments, a macro lens is utilized to enable a camera system to capture images close to the camera aperture. The term macro lens is typically used to refer to optical systems (including compound lenses) that are designed to enable capture of extreme closeup images. In a number of embodiments, a camera module containing a macro lens can be positioned with a field of view of the region below the handheld treatment device. An injector device incorporating an imaging system including a camera with a macro lens in accordance with an embodiment of the invention is illustrated in FIG. 26C. A challenge that can be experienced with macro lenses is that they often have limited depths of field. Accordingly, handheld treatment devices in accordance with many embodiments of the disclosure will often utilize imaging systems incorporating a macro lens in combination with a stabilizing feature (e.g. a stabilizing foot similar to the stabilizing feet of the handheld treatment devices described above with reference to FIGS. 1A to 1I) and/or an autofocus mechanism.

In several embodiments, the camera system captures color images (e.g., using an image sensor configured with a Bayer color filter pattern). In many embodiments, color images are captured using color filter patterns that include twice as many red pixels as blue pixels or green pixels (e.g., a RGRB Bayer-like filter pattern). In a number of embodiments, the image sensor is also configured to capture image data in the near-infrared spectrum (e.g., by not including an IR cut filter in the optical system). In certain embodiments, a monochrome image sensor is utilized to capture black and white images. In various embodiments, color filters are utilized to enable the capture of monochrome images in specific spectral bands including (but not limited to) a red color channel, near-infrared wavelengths, and/or an extended color spectral band including visible and near-infrared wavelengths. Specific embodiments also utilize image sensors configured to perform multispectral imaging. In a number of embodiments, the optical system of the camera can also include a polarizing filter to enable imaging of polarized light. As can readily be appreciated the specific spectral bands and/or number of channels imaged by an imaging system is largely dependent upon the requirements of specific applications in accordance with embodiments of the disclosure. Furthermore, the specific image sensors and/or spectral filters described above can be utilized in any of the imaging systems described herein including (but not limited to) the imaging systems described with reference to FIGS. 1D-1I and 26A-26C.

### Machine Vision Systems

Handheld treatment devices in accordance with many embodiments of the disclosure utilize machine visions systems to identify features of interest on the skin of a user and/or control application of a treatment. In several embodiments, the machine vision system acquires image data using an imaging system such as, but not limited to, any of the imaging systems described above. The machine vision system can process the image data in real time to identify regions that contain features of interest. In many embodiments the features of interest are dermal conditions (e.g., acne lesion) and the machine vision system both detects and classifies the detected conditions. As is discussed further below, the ability to classify dermal conditions can enable the applications of different treatments. In a number of embodiments, the machine vision system utilizes information regarding detected dermal conditions to guide treatment. In certain embodiments, the machine vision system generates feedback via a user interface to guide the user in the manual initiation of a treatment. In several embodiments, the machine vision system utilizes information concerning the detected features to automatically initiate application of a treatment when the handheld treatment device is positioned appropriately.

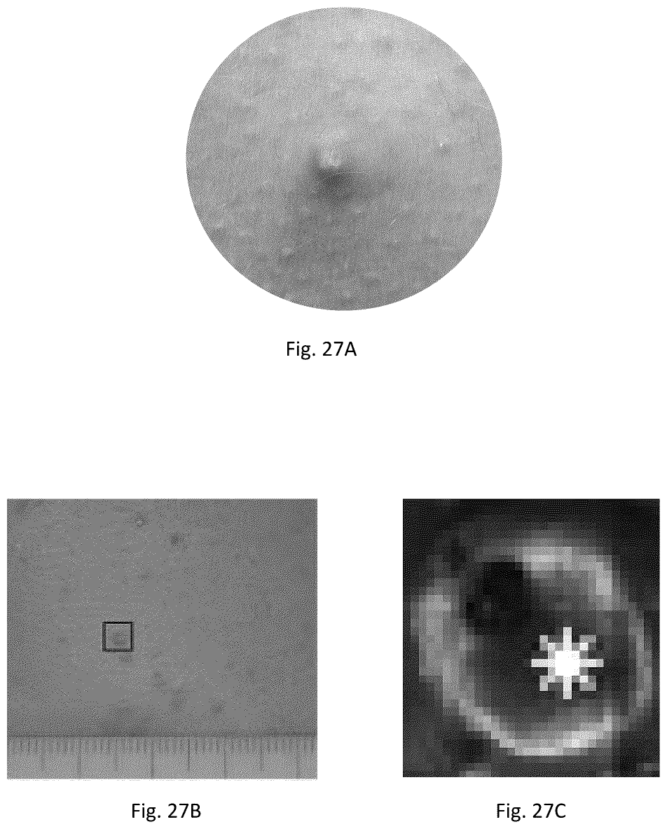

Image data acquired by an imaging system forming part of a machine vision system of a handheld treatment device is conceptually illustrated in FIG. 27A. In the illustrated embodiment, the image data is a color image dewarped to remove distortions introduced by the optics of the camera used to capture the image data. As is discussed further below, machine vision systems in accordance with many embodiments of the disclosure can detect the presence of a dermal condition within the image. Once detected, a variety of processes can be performed by the machine vision system including (but not limited to) classification of the dermal condition, tracking of the detected dermal condition, and targeting of application of a treatment. Within FIG. 27A and throughout the various examples of machine vision processes (see FIGS. 27A to 29B), the dermal condition detected is an acne lesion. It is to be understood that the acne lesion is utilized as an example of a dermal condition and that a variety of dermal conditions can be detected and treated in accordance with the various embodiments of the invention. Accordingly, the devices and methods described herein can be utilized to detect and treat any dermal condition that can be detected via machine vision learning and treated via intradermal or subdermal fluidic injection. A dermal condition can be a skin ailment, a lesion (e.g., acne lesion), dermal injury, keloid, wrinkle, dermal abnormality, discoloration, or any other dermal condition that is detectable and capable of being treated by a treatment system as described herein.

While the image shown in FIG. 27A is a color image, machine vision systems in accordance with many embodiments of the invention can acquire image data in any of a variety of spectral bands including (but not limited to) acquiring image data in multiple spectral bands. FIGS. 27B and 27C conceptually illustrate image data that can be acquired in the visible light and near infrared spectrums. FIGS. 27B and 27C are reproduced from Manfredini, M., et al. “In vivo monitoring of topical therapy for acne with reflectance confocal microscopy.” Skin Research and Technology 23.1 (2017): 36-40, the disclosure of which is incorporated by reference herein in its entirety. FIG. 27B illustrates and image of an acne lesion. FIG. 27C is an image generated using reflected near infrared wavelengths of light. The fiducial marker in FIG. 27C indicates a pore in the skin and the image itself captures information concerning the underlying structure of the pilosebaceous unit. As is discussed further below, infrared wavelengths can penetrate the skin of the subject enabling an imaging system that capture image data in the infrared spectrum to capture information concerning the underlying structure of the pilosebaceous unit. The degree to which infrared light penetrates skin is dependent on interactions of the infrared light with molecules such as water and hemoglobin. In many embodiments, a polarized light illumination source can be utilized in combination with an imaging system having a linear polarizing filter to image features of acne lesions including (but not limited to) features of a pilosebaceous unit.

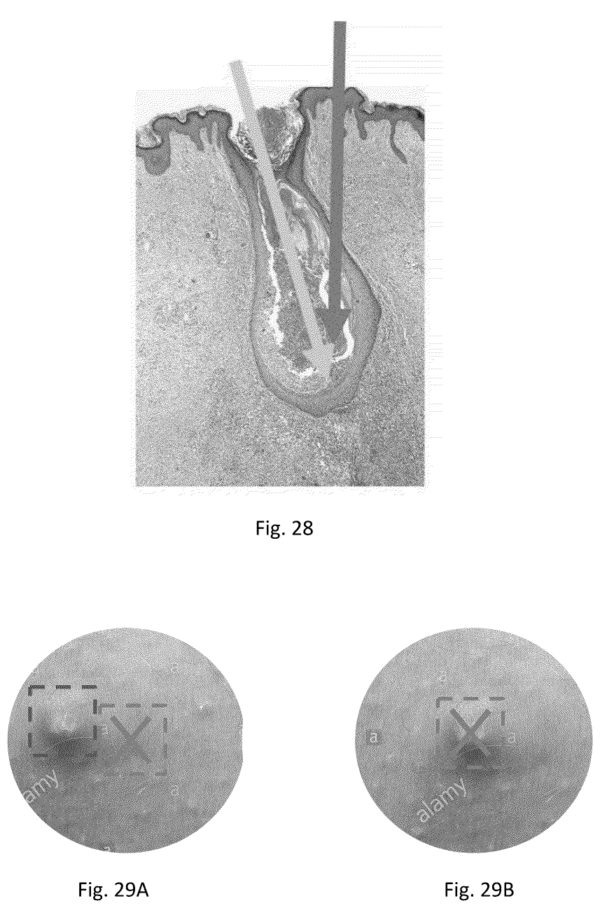

Information concerning the underlying structure of the pilosebaceous unit can be utilized in the targeting of the administration of treatment using techniques including (but not limited to injection). For example, the injection site and trajectory of the need may be dependent on various particularities. In some instances, when an acne lesion is cystic or papular, treatment can be administered via a injection in the center of the acne lesion, where the trajectory of the injection follows the angle and path of a hair follicle contained within the pilosebaceous unit. An injection trajectory in the center of an acne lesion following the angle and path of a hair follicle is conceptually illustrated in FIG. 28 and shown as a yellow arrow. By following the follicle, trauma to surrounding skin and atrophy of the surrounding tissue can be reduced. In some instances in which the injection administers an anti-inflammatory agent, anti-inflammatory potency can be increased by delivery of the anti-inflammatory agent to the bulb of the hair follicle. The red arrow in FIG. 28 indicates an injection into the bulb of the hair follicle that is directly downward through the skin of the user and is more likely to cause trauma to surrounding tissue.

While specific treatments for acne lesions that cystic or papular are described above with respect to FIG. 28, machine vision systems in accordance with many embodiments of the disclosure possess the capability to classify detected acne lesions. In some instances, when an acne lesion is detected that is pustular, then the machine vision system can administer an injection in a location adjacent the visible pore of the acne lesion to avoid pus filling the pilosebaceous unit from diluting the delivered medication. In several embodiments, the machine vision system directs the injection in a trajectory parallel to the pilosebaceous unit, which can enhance efficacy and minimize skin trauma. It should be understood that the injection site and trajectory described are potential examples and should not be construed as limiting the injection site and trajectory for cystic, popular, or pustular acne lesions.

A process that can be utilized by a machine vision system in accordance with an embodiment of the invention to detect a skin condition and administer a treatment in accordance with various embodiments of the disclosure is conceptually illustrated in FIGS. 29A and 29B. In this particular example, the process involves initially detecting an acne lesion, which is indicated in FIG. 29A by a blue bounding box.

In several embodiments, a skin condition is detected by identifying regions of interest within an image that are likely to contain the skin condition. In a number of embodiments, a classifier can be utilized to determine the type of skin condition contained within a region of interest. As noted in the example above, classification of an acne lesion can determine the manner in which a treatment is administered by the machine vision system using the handheld treatment device. In the illustrated example, the skin condition is an acne lesion visible in FIG. 29A and is determined to be a papular lesion. In some instances, the machine vision determines the popular lesion is to be treated via injection of medication into the pore of the acne lesion. The machine vision system can track the acne lesion and compares the location of the acne lesion to a current target injection site of the handheld treatment device. In FIG. 29A the region of interest containing the acne lesion is adjacent the target injection site of the handheld treatment device, which is indicated by the red bounding box. FIG. 29B conceptually illustrates the user moving the handheld treatment device so that the acne lesion is located within the target injection site. In several embodiments, the machine vision system provides feedback via a user interface instructing the user to administer an injection. In a number of embodiments, the machine vision system automatically administers the injection.

Machine vision systems in accordance with certain embodiments of the invention can integrate signals for additional sensors within a handheld treatment device and/or other devices. In several embodiments, the machine vision system administers treatments via injection and the set of one or more injection needles utilized to administer the treatment is monitored using a force or displacement sensor. Where force or displacement sensor information is available, the machine vision system can utilize the sensor information to control the depth of the injection.

In certain embodiments, the machine visions system is capable classifying the location of skin in determining depth of injection. Location of skin on a user's body can influence injection depth (e.g., forehead is typically shower than skin on a user's back). Location of skin can be determined based upon one or more of user input, image data, and/or inertial measurements from an inertial measurement unit of the orientation of the handheld treatment device relative to gravity. Machine vision system can also utilize information including (but not limited to) a classification of a stage of a dermal condition to influence the depth of an injection. In several embodiments, the machine vision system can perform classification based upon one or more of color, height relative to plane of surrounding skin and/or diameter of a dermal condition. As can readily be appreciated any of a variety of machine vision classifications, sensor inputs obtained prior to injection and/or sensor inputs obtained during injection can be utilized to determine and/or control depth of injection as appropriate to the requirements of specific applications in accordance with various embodiments of the disclosure.

Although a variety of machine vision systems and processes for administering treatments including (but not limited to) injection treatments are described above with respect to FIGS. 27A to 29B, any of a variety of machine vision systems incorporating any of a number of different imaging systems can be utilized to acquire image data and perform processes that direct administration of a treatment as appropriate to the requirements of specific applications (including applications involving any of a variety of dermatological conditions) in accordance with various embodiments of the disclosure. Machine visions processes and processing systems that can be utilized to implement machine vision processes in accordance with various embodiments of the invention are discussed further below.

### Machine Vision Processes

Machine vision systems in accordance with various embodiments of the disclosure are capable of detecting features such as (but not limited to) dermal conditions (e.g., acne lesions) on a user's skin for the purpose of administering a treatment. The processes can be performed in real time based upon image data captured at short range as a user manipulates a handheld treatment device incorporating an imaging system.

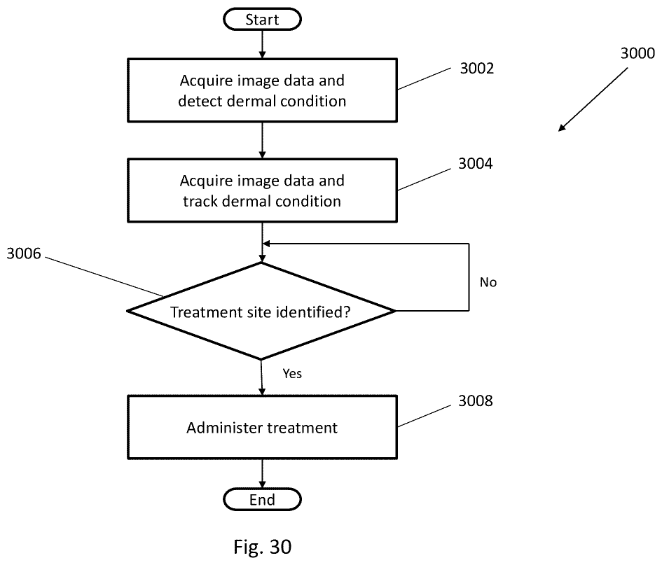

A process for administering a treatment using a handheld treatment device based upon image data is conceptually illustrated in FIG. 30. The process 3000 includes acquiring image data and detecting (3002) a dermal condition. In several embodiments, real time processing is achieved by acquiring addition images and utilizing a tracking process to track (3004) the location of the dermal condition. In this way, the location of a lesion detected in a previous image can be utilized to predict the location of the lesion in the newly acquired image. By constraining the search for the condition, computational efficiencies can be attained that enable the location of the acne lesion to be determined in real time.

As discussed above, a condition may be visible within the field of view of the machine vision system but not be positioned in a location in which a treatment can be effectively administered. In addition, the treatment location itself may be determined based upon a classification of the condition. Accordingly, a determination (3006) is made concerning whether the location of the dermal condition and/or its orientation relative to a handheld treatment device is appropriate for the administration of a treatment appropriate to the type of condition. When the handheld treatment device is appropriately positioned relative to the condition, then the treatment can be administered (3008). As noted above, the machine vision system can provide an indication via a user interface on the handheld treatment device encouraging the user to manually initiate administration of a treatment. In several embodiments, the machine vision system can initiate the automated administration of a treatment.

When the position of the handheld treatment device is not appropriate to administer a treatment, the handheld device can continue to track (3004) the location of the dermal condition. In several embodiments, the machine vision system can provide feedback (e.g., visual and/or audio feedback) via a user interface to guide the user in the manipulation of the handheld treatment device with respect to the detected dermal condition. In this way, the handheld treatment device can encourage the user to position the handheld treatment device in an orientation in which it is appropriate to administer a treatment.

While specific machine vision processes are described above with respect of FIG. 30, any of a variety of machine vision process can be utilized including processes that are modified to accommodate different imaging systems, illumination sources, and/or treatment modalities as appropriate to the requirements of specific applications in accordance with various embodiments of the invention. Specific processes that can be utilized to perform detection, tracking and/or classification in machine vision processes such as (but not limited to) the machine vision processes described above with respect to FIG. 30 are discussed further below.

### Machine Vision Process Incorporating Machine Vision Models

Machine vision systems incorporated within handheld treatment devices in accordance with various embodiments of the invention can utilize machine vision models to perform detection, tracking and/or classification of dermal conditions. In a number of embodiments, a neural network such as the Single Shot MultiBox Detector described in Liu, Wei, et al. “Ssd: Single shot multibox detector.” European conference on computer vision. Springer, Cham, 2016 (incorporated by reference above) is utilized. However, it should be readily appreciated that detection, tracking and/or classification in a machine vision process in accordance with various embodiments of the disclosure can be performed using any of a variety of heuristics and/or machine learning models adapted for use in image processing applications including (but not limited to) convolutional neural networks (CNNs) such as Alexnet, ResNet, VGGNet, and/or Inception. As can readily be appreciated, the specific machine learning models that are utilized are largely dependent upon the requirements of specific applications.

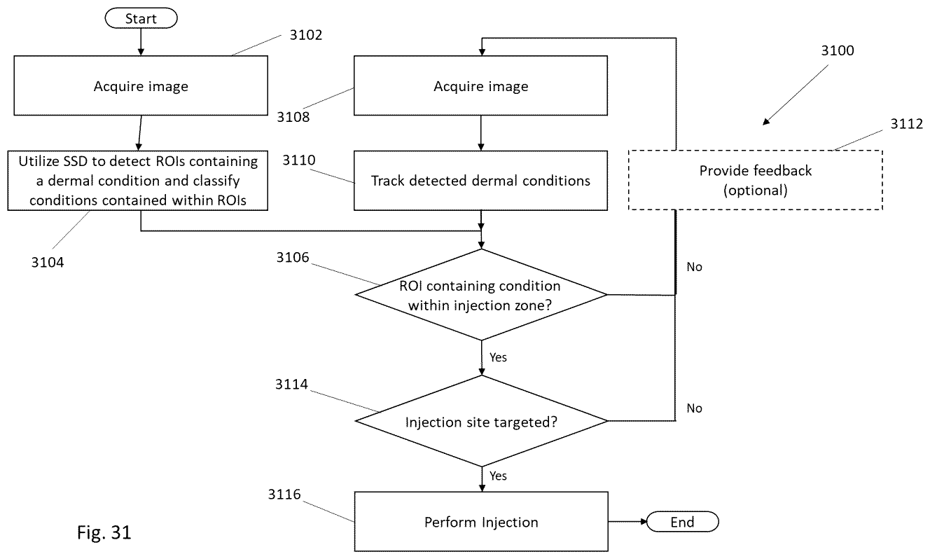

A machine vision process incorporating the use of a single shot multibox detector (SSD) machine learning model to perform detection and classification of dermal conditions in accordance with various embodiments of the disclosure is conceptually illustrated in FIG. 31. The process 3100 includes acquiring (3102) an image and performing detection of a dermal condition using an SSD detector. An SSD detector utilizes a convolutional neural network that is trained to accept an image patch (e.g. a 200×200 pixel image patch) and extract features that can be utilized to both: i) determine the likelihood that specific size and aspect regions of interest contain a dermal condition; and ii) classify detected dermal conditions. In several embodiments that involve classification of acne lesions, the classifier can determine the likelihood that detected lesions are a cystic acne lesion, a papulopustular acne lesion, an open comedome, and/or a closed comedome.

When a lesion is detected and classified, a desired injection site and/or injection orientation can be determined. Based upon this determination, the process 3100 can evaluate (3106) whether the region of interest (ROI) containing the detected dermal condition is within an injection zone in which an injection can potentially be administered by the handheld treatment device. While the discussion of FIG. 31 refers to treatment via injection, it should be readily appreciated that similar processes can be utilized in combination with alternative treatment modalities.

When the detected dermal condition is not located within an injection zone in which an injection can potentially be administered by the handheld treatment device, the machine vision process can continue to acquire (3108) images and track (3110) the detected image until the detected lesion is located within an injection zone. In many embodiments, the machine vision process can provide (3112) feedback (e.g., audio, haptic, tactile, and/or visual feedback) via the handheld treatment device and/or another device such as (but not limited to) a mobile computing device (e.g., mobile phone, tablet computer, and/or laptop computer in communication with the handheld treatment device) to assist the user in positioning the handheld treatment device in an appropriate orientation. In a number of embodiments, a camera is utilized to capture live video of the user manipulating the handheld treatment device (e.g., via a front facing camera on a mobile phone) and feedback user interface devices are displayed on the live video to direct the user. In many embodiments, the handheld treatment device includes an array of piezoelectric devices that can provide vibrational haptic feedback in different positions on the surface of the handheld treatment device that can provide guidance regarding the manipulation of the handheld treatment device by the user. As can readily be appreciated, the specific manner in which the machine vision process provides feedback to the user is largely determined by the requirements of a specific application.

When the machine vision process 3100 determines (3106) that a detected dermal condition is located within the injection zone of a portable treatment device, a determination can be made concerning whether an appropriate injection site is currently being targeted by the handheld treatment device. In several embodiments, the determination is based upon a position(s) in which one or more needles will penetrate the user's skin given the current orientation of the handheld treatment device. In a number of embodiments, the determination is based upon a trajectory with which one or more needles will penetrate the user's skin given the current orientation of the handheld treatment device. The process continues to acquire and analyze imaged data until the target is acquired.

When a target is acquired, the machine vision process can cause the injection to be performed (3116). In several embodiments, the machine vision process provides an indication (e.g. audio, tactile and/or visual indication) to the user to manually initiate the injection. In many embodiments, the machine vision process automatically initiates the injection.

While a variety of machine vision processes that utilize machine learning models to administer treatments are described above with respect to FIG. 31, any of a variety of machine learning processes that utilize heuristics and/or different classes of machine model such as (but not limited to) neural networks, convolutional neural networks, recurrent neural networks, support vector machines and/or cascades of classifiers can be utilized as appropriate to the requirements of specific applications in accordance with various embodiments of the disclosure. Various processes that can be utilized by machine vision systems to acquire image data, determine injection targets, and/or perform injections in accordance with different embodiments of the disclosure are discussed further below.

### Image Data Acquisition

Image data acquisition processes that can be utilized in accordance with various embodiments of the disclosure typically depend upon the specific image sensors and/or imaging modalities utilized to acquire the image data. In many embodiments, image data is acquired using a camera having an optical system including a lens or compound lens and an image sensor (e.g., a CMOS image sensor). In a number of embodiments, the imaging system captures an image that include geometric and/or photometric distortions that can be intentional (e.g., due to optical prescriptions of the lens system) and/or unintentional (e.g., defects in the optics and/or image sensor). Accordingly, image acquisition processes in accordance with a number of embodiments utilize a dewarping transformation and/or image normalization to transform captured image data into an acquired image that can be provided to subsequent image processing operations within a machine vision process.

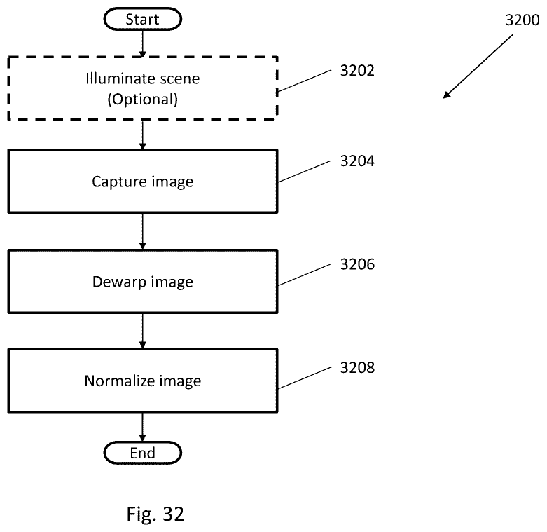

A process for acquiring image data in accordance with an embodiment of the invention is conceptually illustrated in FIG. 32. The process 3200 can commence with the illumination (3202) of the scene being imaged using an illumination source. As discussed above, illumination sources such as (but not limited to) infrared and/or linear polarized light sources can be utilized to image features that are pronounced when thus illuminated. The process 3200 includes capturing (3204) image data. The image data is dewarped (3206). In a number of embodiments, the dewarping is performed based upon calibration data. The dewarped image can also be photometrically normalized (3208) utilizing photometric calibration data. The resulting acquired image can then be subject to additional transformations (e.g., edge enhancement and/or high pass filtering) prior to being provided as an input to machine vision processes such as (but not limited to) feature detection processes.

While various image data acquisition processes are described above with respect to FIG. 32, any of a variety of image data acquisition processes appropriate to the requirements of particular imaging systems and/or machine vision processes can be utilized as appropriate to the requirements of specific applications in in accordance with various embodiments of the disclosure. Processes that can be utilized within machine vision processes to identify injection site targets in accordance with various embodiments of the invention are discussed further below.

### Identification of Injection Site Targets

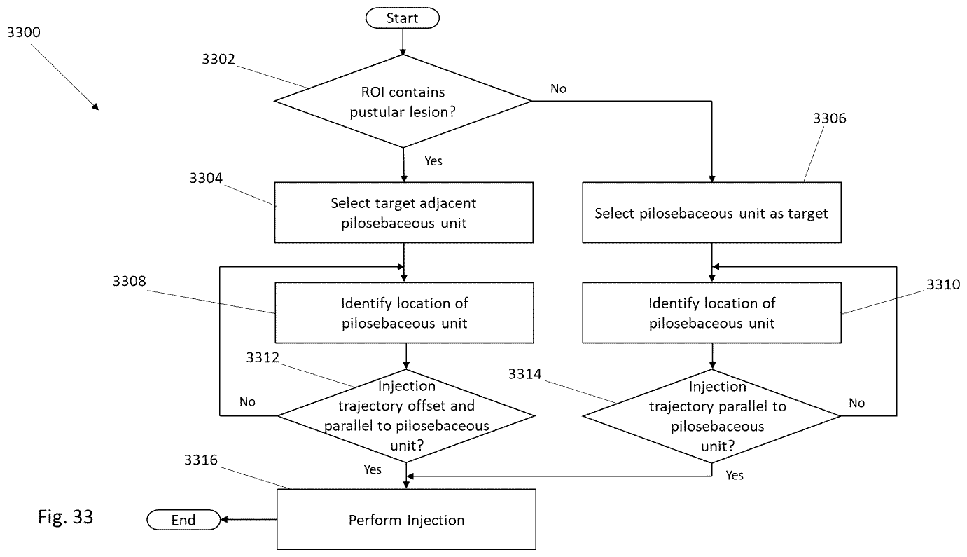

The response of various types of dermal conditions can depend upon the site in which a treatment is administered. Accordingly, machine vision processes in accordance with many embodiments of the invention target treatment sites in a manner that is dependent upon classification of a particular lesion and/or feature. The example below is focused on classification of an acne lesion, but it is to be understood that any dermal condition that can be treated by alternative injection methods dependent upon a classification can utilize an injection process as described in FIG. 33.

A process that can be utilized by a machine vision system to determine injection site targets for acne lesions in accordance with various embodiments of the disclosure is shown in FIG. 33. The process 3300 includes determining (3302) whether a particular region of interest in which an acne lesion is detected contains a pustular lesion. When the lesion is a pustular lesion, the process 3300 targets (3304) an injection site adjacent the pilosebaceous unit. When the lesion is not a pustular lesion, the process 3300 targets (3306) the pilosebaceous unit as the injection site.

Both potential targets require knowledge of the location of the pilosebaceous unit. Accordingly, the pilosebaceous unit is identified (3308, 3310) irrespective of the type of lesion. When the lesion is a pustular legion, the orientation of the handheld treatment device is monitored until a determination (312) is made that the orientation will result in an injection trajectory that is offset and parallel to the pilosebaceous unit. At which point, the process causes the injection to be performed (3316). As noted above, the process can provide an indication to the user to initiate the injection and/or automatically initiate the injection. When the lesion is not a pustular legion, the orientation of the handheld treatment device is monitored until a determination (3314) is made that the orientation will result in an injection trajectory that enters the pilosebaceous unit through the pore and is parallel to the pilosebaceous unit. At which point, the process causes the injection to be performed (3316).

While specific processes are described above for selecting treatment site targets based upon a classification performed within a machine vision process with reference to FIG. 33, any of a variety of processes can be utilized that incorporate a variety of image data, utilize any of a variety of machine vision classification techniques, and/or acquire injection site targets in any of a variety of ways appropriate to the requirements of specific applications in accordance with various embodiments of the disclosure.

### Processes for Controlling Injection

When a decision is made to initiate an injection, handheld treatment devices in accordance with various embodiments of the disclosure are capable of administering an injection to a depth appropriate to the specific treatment being administered. As noted above, the specific depth can be dependent upon the location of the body and/or factors including (but not limited to) the specific treatment being administered. In a number of embodiments, the depth of the injection is determined based upon sensor information received during the injection process.

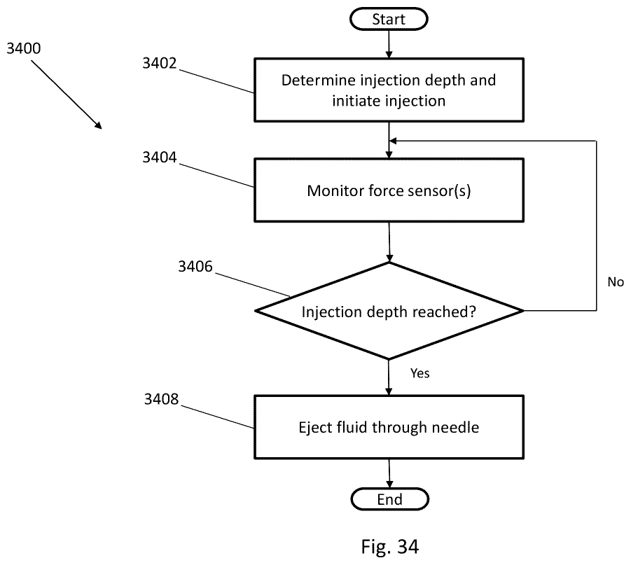

A process for automatically performing an injection using a needle or microneedle in accordance with an embodiment of the invention is illustrated in FIG. 34. The process 3400 may include determining an initial injection depth and commences with the initiation (3402) of the injection. During the injection, force and/or displacement sensors are monitored (3404) and information derived from the sensors is utilized to determine whether an appropriate depth is reached to stop further penetration of the one or more needles or microneedles and/or commence delivery of a treatment (e.g., injection). In the illustrated embodiment, the treatment includes ejection of fluid through the needle or microneedle. As can readily be appreciated any of a variety of treatments can be administered using a process similar to the processes described with reference to FIG. 34 including (but not limited to) any of the treatment modalities described above. Furthermore, any of the various machine vision processes can be utilized in alone or in combination within machine vision systems implemented in accordance with embodiments of the disclosure. A variety of computational platforms that can be utilized to implement machine vision systems in accordance with various embodiments of the disclosure are discussed further below.

### Machine Vision Processing System

A machine vision system utilized within a handheld treatment device in accordance with various embodiments of the disclosure typically utilizes a processing system including one or more of a CPU, GPU and/or neural processing engine. In a number of embodiments, image data is captured and processed using an Image Signal Processor and then the acquired image data is analyzed using one or more machine learning models implemented using a CPU, a GPU and/or a neural processing engine. In several embodiments, the machine vision processing system is housed within the handheld treatment device. In a number of embodiments, the machine vision processing system is housed separately from and communicates with the handheld treatment device. In certain embodiments, the machine vision processing system is connected to the handheld treatment device via a cable. In various embodiments, the machine vision processing system communicates with the handheld treatment device via a wireless connection. In several embodiments in which the machine vision processing system is separate from the handheld treatment device, the handheld treatment device includes an imaging system and a processing system that handles the acquisition of image data. In many embodiments, the processing system also encodes the acquired image data and transmits the encoded image data to the machine vision processing system. In certain embodiments, the machine vision processing system is implemented as a software application on a computing device such as (but not limited to) mobile phone, a tablet computer, a wearable device (e.g., watch and/or AR glasses), and/or portable computer.

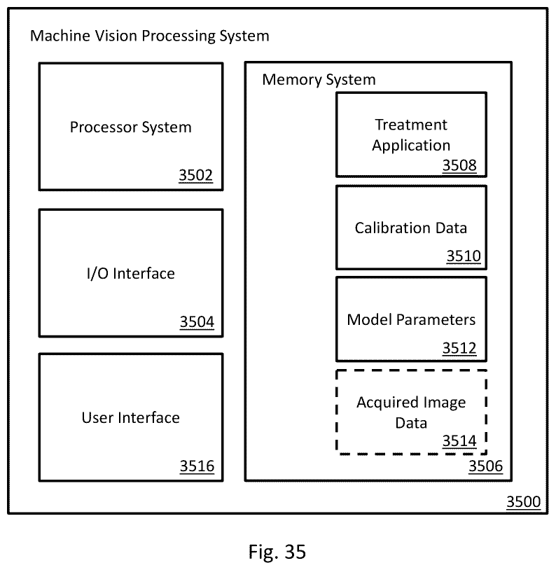

A machine vision processing system in accordance with various embodiments of the disclosure is illustrated in FIG. 35. The machine vision processing system 3500 includes a processor system 3502, an I/O interface 3504, a sensor system 3505 and a memory system 3506. As can readily be appreciated, the processor system 3502, I/O interface 3504, sensor system 3505 and memory system 3506 can be implemented using any of a variety of components appropriate to the requirements of specific applications including (but not limited to) CPUs, GPUs, ISPs, DSPs, wireless modems (e.g., WiFi, Bluetooth modems), serial interfaces, depth sensors, IMUs, pressure sensors, ultrasonic sensors, volatile memory (e.g., DRAM) and/or non-volatile memory (e.g., SRAM, and/or NAND Flash). In the illustrated embodiment, the memory system is capable of storing a treatment application 3508. The treatment application can be downloaded and/or stored in non-volatile memory. When executed the treatment application is capable of configuring the processing system to implement machine vision processes including (but not limited to) the machine vision processes described above and/or combinations and/or modified versions of the machine vision processes described above. In several embodiments, the treatment application 3508 utilizes calibration data 3510 stored in the memory system 3506 during image acquisition to perform processing including (but not limited to) dewarping and photometric normalization of digitally captured images received via the I/O interface 3504 from one or more image acquisition systems (not shown), such as (but not limited to) a camera, a depth camera, a near-IR camera, and/or any other type of imaging system capable of capturing image data using an imaging sensor. In certain embodiments, the treatment application 3508 utilizes model parameters 3512 stored in memory to process acquired image data using machine learning models to perform processes including (but not limited to) detection, tracking, classification, and/or treatment targeting. Model parameters 3512 for any of a variety of machine learning models including (but not limited to) the various machine learning models described above can be utilized by the treatment application. In several embodiments, acquired image data 3514 is temporarily stored in the memory system during processing and/or saved for use in training/retraining of model parameters.

In a number of embodiments, the machine vision processing system also includes a user interface. In several embodiments, the user interface can any of a variety of input and/or output user interface modalities including (but not limited to) buttons, audio devices, visual display devices (e.g., LEDs and/or displays). In certain embodiments, the machine vision processing system communicates with an external device (e.g., a mobile phone) to display a user interface. As can readily be appreciated, the specific user interface and/or user interface input and output modalities is largely dependent upon the requirements of specific applications in accordance with various embodiments of the disclosure.

While specific machine vision processing systems are described above with reference to FIG. 35, it should be readily appreciated that machine vision processes and/or other processes utilized in the provision of treatment via handheld treatment devices in accordance with various embodiments of the disclosure can be implemented on any of a variety of processing devices including combinations of processing devices. Accordingly, handheld treatment devices in accordance with embodiments of the disclosure should be understood as not limited to specific imaging systems, illumination systems, machine vision processing systems, treatment systems and/or injection systems. Handheld treatment devices can be implemented using any of the combinations of systems described herein and/or modified versions of the systems described herein to perform the processes, combinations of processes, and/or modified versions of the processes described herein.

### Applications of Liquid Delivery

The various embodiments of intradermal or subdermal fluid delivery systems can be utilized in a number of applications that require liquid delivery into the skin. In certain embodiments, a fluid delivery system is used for delivery of medication or supplement into the skin. In certain embodiments, triamcinolone (Kenalog) is utilized within a fluid delivery system. In certain embodiments, hyaluronic acid is utilized within a fluid delivery system. In certain embodiments, collagen or a collagen stimulating agent is utilized within a fluid delivery system.

Triamcinolone is a glucocorticoid use to treat various skin ailments, including (but not limited to) acne, eczema, dermatitis, allergies, and rash. Triamcinolone can reduce swelling, itching, and redness.

Treatment of an acne lesion can reduce the swelling and redness within 12 hours with single dose at a volume of 0.01 mLs to 0.20 mLs and at a concentration between 0.5 mg/mL and 10 mg/mL. Accordingly, a solution containing triamcinolone can be contained within fluid container (e.g., syringe or cartridge), as described herein. The triamcinolone-containing container can be utilized within an injector system with a microneedle. The needle or microneedle can penetrate the skin the requisite amount for intralesion delivery (e.g., intradermal or subdermal delivery at the site of the lesion). The injector system can inject the triamcinolone into the lesion as a treatment. The treatment can be performed on multiple times on a single lesion or can be performed on multiple lesions, as needed. In many instances, a single dose will result in substantial clearance of an acne lesion. Similar procedures can be performed on other skin ailments.

Hyaluronic acid is a glycogen that is naturally produced in the skin. Hyaluronic acid injections into the skin can boost the amount of localized skin hyaluronic acid. Benefits of hyaluronic acid include (but are not limited to) mitigating the appearance of aging of skin, reducing wrinkles, reducing inflammation in the skin, and assisting in would healing.

Collagen is protein that is naturally produced in the skin. Collagen injections (or injection of collagen stimulating agents) into the skin can boost the amount of localized skin collagen. Benefits of collagen (or collagen stimulating agent) include (but are not limited to) reducing appearance of scars (especially acne scars), flattening out wrinkles, and filling-in skin depression. Collagen stimulating agents include (but are not limited to) microneedling, vitamin C, proline, glycine, copper, aloe vera, ginseng, and algae.

Various medications and supplements can be combined within the same cartridge for use in intradermal or subdermal fluid delivery system. For instance, one exemplary combination is triamcinolone with collagen (or a collagen stimulating agent).

What is claimed is:

1. A dermal condition treatment system, comprising:
memory comprising a treatment application;
a set of one or more processors; and
a handheld device comprising:
an injection system comprising:
a fluid-filled container, a needle in fluidic connection with the fluid-filled container, and an internal driver system capable of ejecting fluid out of the needle from the fluid-filled container; and
an image acquisition system comprising camera optics;
wherein the memory and the set of one or more processors is in communication with the handheld device;
wherein the set of one or more processors is capable of performing steps via the treatment application, comprising:
acquiring image data using the image acquisition system;
detecting a feature in the acquired image data;
identifying a treatment site using the acquired image data; and
initiating a treatment injection at the treatment site via the injection system.

2. The dermal condition treatment system of claim 1, wherein the injection system performs intradermal or subdermal fluidic injection at the treatment site upon performing the steps of the treatment application.

3. The dermal condition treatment system of claim 1, wherein the camera optics comprises:
a Bayer camera;
a monochrome camera capable of imaging red light;
a monochrome camera capable of imaging an extended color spectral band including visible and near-infrared wavelengths;
a camera capable of imaging near-infrared light;
a camera capable of imaging infrared light;
a camera including a polarizing filter;
a camera capable of capturing a multispectral image; or
a depth camera.

4. The dermal condition treatment system of claim 1, wherein the camera optics comprises:
a macro lens,
telecentric optics, or
periscope optics.

5. The dermal condition treatment system of claim 1, further comprising:
an illumination source capable of activation by the set of one or more processors;
wherein the set of one or more processors is also capable of performing an additional step of activating the illumination source via the treatment application.

6. The dermal condition treatment system of claim 5, wherein the illumination system is selected from the group consisting of:
an infrared light source;
a near-infrared light source; and
a linear polarized light source.

7. The dermal condition treatment system of claim 1, further comprising:
a near-infrared light source capable of being activated by the set of one or more processors via the treatment application;
wherein the image acquisition system comprises at least one camera that is capable of imaging near-infrared light.

8. The dermal condition treatment system of claim 1, further comprising:
a linear polarized light source capable of being activated by the set of one or more processors via the treatment application;
wherein the image acquisition system comprises at least one camera that includes a polarizing filter.

9. The dermal condition treatment system of claim 1, wherein:
the acquired image data comprises a sequence of images;
wherein the detecting the feature in the acquired image data comprises detecting a dermal condition in the sequence of images;
wherein the identifying the treatment site using the acquired image data comprises:
tracking the detected dermal condition using the sequence of images.

10. The dermal condition treatment system of claim 1 further comprising a sensor for monitoring depth of injection, wherein the set of one or more processors is capable of directing the internal driver to control the depth of injection via the treatment application and the sensor.

11. The dermal condition treatment system of claim 1, wherein the set of one or more processors is housed within the handheld device.

12. The dermal condition treatment system of claim 1, wherein the set of one or more processors is housed separately from the handheld device.

13. The dermal condition treatment system of claim 1, wherein the needle is a hollowed microneedle.

14. The dermal condition treatment system of claim 1, wherein the fluid-filled container comprises a syringe.

15. The dermal condition treatment system of claim 1, wherein the fluid-filled container comprises a cartridge.

16. The dermal condition treatment system of claim 1, wherein the fluid within the fluid-filled container comprises a medication or supplement.

17. The dermal condition treatment system of claim 1, wherein the fluid within the fluid-filled container comprises triamcinolone.

18. A dermal condition treatment system, comprising:
memory comprising a treatment application;
a set of one or more processors; and
a handheld device comprising:
an injection system comprising at least one needle, where the injection system is capable of ejecting a liquid through the at least one needle; and
at least one camera capable of communicating with the set of one or more processors;
wherein the memory and the set of one or more processors is in communication with the handheld device;
wherein the set of one or more processors is capable of performing steps via the treatment application, comprising:
acquiring image data using the at least one camera, the image data comprising a sequence of images;
detecting a lesion in the sequence of images;
tracking the detected lesion using the sequence of images;
identifying a treatment site using the sequence of images; and
initiating injection of the liquid into the treatment site using the injection system.

19. The dermal condition treatment system of claim 18, wherein acquiring image data using the at least one camera further comprises:
capturing an image using the at least one camera;
dewarping the captured image; and
normalizing the dewarped image.

20. The dermal condition treatment system of claim 18, wherein one of the at least one camera comprises at least one filter selected from the group consisting of:
a polarizing filter;
a Bayer color filter that filters light on a set of four adjacent pixels such that two of the pixels image Green light, one of the pixels images Blue light, and one of the pixels images Red light;
a Bayer color filter that filters light on a set of four adjacent pixels such that two of the pixels image Red light, one of the pixels images Blue light, and one of the pixels images Green light;
a multispectral filter; and
a color filter that enables capture of a monochrome image in a specific spectral band selected from the group consisting of:
a red color channel;
near-infrared wavelengths; and
an extended color spectral band including visible and near-infrared wavelengths.

21. The dermal condition treatment system of claim 18, further comprising:
an illumination source capable of activation by the set of one or more processors;
wherein the set of one or more processors is also capable of performing an additional step of activating the illumination source via the treatment application.

22. The dermal condition treatment system of claim 18, wherein the handheld device further comprises an illumination system, wherein the illumination system comprises:
an infrared light source;
a near-infrared light source; or
a linear polarized light source.

23. The dermal condition treatment system of claim 18, further comprising:
a near-infrared light source capable of being activated by the set of one or more processors via the treatment application;
wherein one of the at least one camera is capable of imaging near-infrared light.

24. The dermal condition treatment system of claim 18, further comprising:
a linear polarized light source capable of being activated by the set of one or more processors via the treatment application;
wherein one of the at least one camera includes a polarizing filter.

25. The dermal condition treatment system of claim 18, wherein the set of one or more processors is also capable of performing the additional step of classifying the lesion via the treatment application.

26. The dermal condition treatment system of claim 18, wherein the set of one or more processors, via the treatment application, is also capable of performing an additional step of:
determining whether the lesion is a pustular lesion;
when the lesion is determined to be a pustular lesion, selecting a target adjacent pilosebaceous unit of the lesion as the treatment site and initiating an injection at a trajectory offset to the pilosebaceous unit of the lesion.

27. The dermal condition treatment system of claim 26, wherein the set of one or more processors, via the treatment application, is also capable of performing an additional step of:
when the lesion is determined not to be a pustular lesion, selecting a pilosebaceous unit of the lesion as the treatment site and initiating an injection at a trajectory parallel to the pilosebaceous unit of the lesion.

28. The dermal condition treatment system of claim 18, wherein the injection system further comprises at least one force or displacement sensor and is capable of being controlled by the set of at least one processor via the treatment application.

29. The dermal condition treatment system of claim 28, wherein initiating injection of the liquid into the treatment site using the injection system comprises:
determining an injection depth;
monitoring sensor data generated by the at least one force or displacement sensor;
determining whether the injection depth is reached based upon the sensor data; and
when the injection depth is determined to have been reached, controlling the injection system to eject the liquid through the at least one needle.

30. The dermal condition treatment system of claim 18, wherein initiating injection of the liquid into the treatment site using the injection system comprises providing an indication via a user interface, where the indication directs a user to manually initiate the injection.

31. The dermal condition treatment system of claim 18, wherein the set of one or more processors is housed within the handheld device.

32. The dermal condition treatment system of claim 18, wherein the set of one or more processors is housed separately from the handheld device.

33. The dermal condition treatment system of claim 18, wherein the needle is a hollowed microneedle.

34. The dermal condition treatment system of claim 18, wherein the liquid is a medication for treating the lesion.

35. The dermal condition treatment system of claim 18, wherein the liquid is triamcinolone.

36. A lesion treatment system, comprising:
memory comprising an injection application;
a set of one or more processors; and
a handheld device comprising:
an injection system comprising at least one needle and at least one force or displacement sensor, where the injection system is capable of:
ejecting a liquid through the at least one needle; and
being controlled by the set of at least one processor via the injection application;
at least one camera capable of communicating with the set of one or more processors;
wherein the memory and the set of one or more processors is in communication with the handheld device; and
wherein the set of one or more processors is also capable of performing steps via the injection application, comprising:
acquiring image data using the at least one camera, the image data comprising a sequence of images;
detecting a lesion in the sequence of images;
determining whether the lesion is a pustular lesion;
tracking the detected lesion using the sequence of images;
when the lesion is determined to be a pustular lesion, selecting a target adjacent a pilosebaceous unit of the lesion as a treatment site and controlling the injection system to initiate an injection of the liquid into the treatment site at a trajectory offset to the pilosebaceous unit of the lesion;
when the lesion is determined not to be a pustular lesion, selecting a pilosebaceous unit of the lesion as a treatment site and controlling the injection system to initiate injection of the liquid into the treatment site at a trajectory offset to the pilosebaceous unit of the lesion;
determining an injection depth;
monitoring force or displacement sensor data generated by the at least one force or displacement sensor;
determining whether the injection depth is reached based upon the force or displacement sensor data; and
when the injection depth is determined to have been reached, controlling the injection system to eject the liquid through the at least one needle.

37. The lesion treatment system of claim 36, wherein acquiring image data using the at least one camera further comprises:
capturing an image using the at least one camera;
dewarping the captured image; and
normalizing the dewarped image.

38. The lesion treatment system of claim 36, wherein one of the at least one camera comprises at least one filter selected from the group consisting of:
a polarizing filter;
a Bayer color filter that filters light on a set of four adjacent pixels such that two of the pixels image Green light, one of the pixels images Blue light, and one of the pixels images Red light;
a Bayer color filter that filters light on a set of four adjacent pixels such that two of the pixels image Red light, one of the pixels images Blue light, and one of the pixels images Green light;
a multispectral filter; and
a color filter that enables capture of a monochrome image in a specific spectral band selected from the group consisting of:
a red color channel;
near-infrared wavelengths; and
an extended color spectral band including visible and near-infrared wavelengths.

39. The lesion treatment system of claim 36, further comprising:
an illumination source capable of activation by the set of one or more processors;
wherein the set of one or more processors is also capable of performing an additional step of activating the illumination source via the injection application.

40. The lesion treatment system of claim 36, wherein the handheld device further comprises an illumination system, wherein the illumination system comprises:
an infrared light source;
a near-infrared light source; or
a linear polarized light source.

41. The lesion treatment system of claim 36, further comprising:
a near-infrared light source capable of being activated by the set of one or more processors via the injection application;
wherein one of the at least one camera is capable of imaging near-infrared light.

42. The lesion treatment system of claim 36, further comprising:
a linear polarized light source capable of being activated by the set of one or more processors via the injection application;
wherein one of the at least one camera includes a polarizing filter.

43. The lesion treatment system of claim 36, wherein the set of one or more processors, via the injection application, is also capable of performing the additional step of classifying the lesion.

44. The lesion treatment system of claim 36, wherein the set of one or more processors is housed within the handheld device.

45. The lesion treatment system of claim 36, wherein the set of one or more processors is housed separately from the handheld device.

46. The lesion treatment system of claim 36, wherein the needle is a hollowed microneedle.

47. The lesion treatment system of claim 36, wherein the liquid is a medication for treating the lesion.

48. The lesion treatment system of claim 36, wherein the liquid is triamcinolone.
